SEQUENCE LISTING (74 sequences)

SEQ ID NO: 1 - DNA, 4439 bp, Triticum aestivum
atggggcacagcgcgaggaagaagaagaagaagggcggcgccgggcgcaaggcggccaaggaccacgccgcgcagctcgagggcgaccagaccgcgctcacggacgagctcacggcgctggctgcaatttttctcgaggacttcaaaataacttcacaatcacctcacacccgatttagcatatgtatcaggccttactctgacggcatgggctttggagatttaaatgtttcggctattcttgatgtaatttgctttgctggttacccccacaagtgcccaaagttgcgaatcatacctgagaaaaacttgtgtaaagaagatgctgatcggctactttcccttctttctgaccaggcaaacatttattcccgagaagggcgtgttatgattttcgatttggtagaggctgcccaagaattcctgtcagaaattgctccagctactgattcaacgagtactgctcctcacttaggttcaagtacaatacaggaaataactgacgcagatgtgaatgctagccttgacagtggtccttatcctggaatcttttacatctataactcattcgatttgtatggtcaactatatgaagacaatagttggcaaaggcaaggtttcgacccaacaactgataatgccaggaaaaatattggatctcaagtcaattcaaatgttagaagcaagaggaaaacagtcgacgagaaatcccgtttttcagctgataagatcaatgctgcaaaaagttcatctcaggataatgctgagcatgccatgaagcatggtgttgtacgagaggtagttccaagtttacctgctgttgcagaggaaactgataatgacagcaaaactttgtccacaagcaacggaggaggtatggcagatactccagagaggagtttcagcagtgtacatgaatccgaggactctgaccttgcagatgacggttggaatgatgcagattctgctccagactctggctcttcaaatgcgccatctcatgtttcagacatgtttgatgatgcttcgcaaaataagaaaagggacttaattctggtacacttgcttcggttagcctgtgcgtcaaaagattctctttcggctgctttgccagtaatatcatcggagttatgcaacataggggttctttctgaatgggctaagcaattaatttctgaatctcctgctgtttttggggaaacttttgatcatgtttttgggcagcaaatgatatcttcagagtgctctctattttggagggctgacaattcctcatctaggccaaattcccgttatttaaatgattttgaagagctccgttcacttggtcaggggggctttggccgtgtggcattgtgtaaaaacaagctcgatggacgccaatatgctgtaaagaagatacgcctcaaggataaaagtcctcaagtgaacgaaaagattctaagagaagttgccacactttcacgactgcagcatcagcatgttgttcggtactaccaggcatgggttgaaactgaatatggtcaacgtcatgttgtgaacactggggggtcccgcactgctgagagctctatgtatagtttcgatgagatcagcttatcagacgcaggtgctggaaataagcaggaatccacgtacttgtacatccaaatggagtattgtcctagaaccctgcgacaggactttgagacatatagttcatctttcaatgtcgaccatgcatggcacctatttcgacaaatcgtggaaggcctagcacatgttcatagtcaaggcatcatacaccgggacttgacacctagcaacatattttttgatgtgcgcaacgatatcaaaattggtgattttggtcttgccaaatttctaaagctggagcagctggatcatgaccaatatattcctactgaaggaatgggcgtttcaatggatggaacaggtcaagtaggcacctatttttatactgcaccagaggtagaacagaagtggccacagataaacgaaaaggttgacatgtacagtgcgggtgtgatattctttgagctttggcatccattttcaacagcaatggaaagacatcttgttctcactgatcttaaacagaaaggcgagtctccaatgtcatggtcaacacaatttcctggtcagtcaaatctattaaaacgcttactgtgtccaagcccgtccgaacgtccatctgcgattgagcttttgcaaaatgacttgcctcctcggatggaggacgagtggttaaatgatgtacttagaatgatacagacaccagaagacacttacgtttatgatcgagttatatctacaatatttaatgaagatcgactggtcgccaaaatgcaatgtcagcatgaaagcagtaagaaatccacttgtaaaaatgataacagtgaactcttggattccattattgaggttagcaaagaggtttttaagcgacattgtgctaaaaggttccagatatcacccttgcatacattggatgggaaatttactgaaaatagtggacagacagtgaagatcctaacgcaaggaggagagatgctagaactttgctatgagcagcgaacgccatttgttatgaacgttgctgctaaccagttatcatcatgtaagcgttatgaaatatcatgggttcacagaagagcagttggccattcaactccttatcgttttcttcagggtgattttgacatcattggaggttcttcaccaataacacaggccgaagttatcaaggtagctttggaccttgtgagacgtttttacaattcgaaggcaattgttattcggctgaatcacagcaaactcgctgaagcagtttgttcctgggcaggagttcctcaggaacgaagacagaatgttgctgagtttctatcttctacgcttgttcaatattgcccaaataaggcggatcgtaagtcacagtggagtttgattcgagggcaactattacaggatcttcgtctttctgaagaagttgttgagaagctacataaagcagatcaacgtttttgtggatctgcagatctagtacttgctagattaagagggactcttttctatgataaatctgcttgcaaggctcttgatgatctctccactttcctcaaatatttgaggatatggtcagttgaagaacatattgctattgacgtacttatgcctccttcagactattactacacagatatgttttttcagatatactcaaaagaaggtaatcctgtgttgagttcccatgaaaagttgcttgctgttggaggacgatatgatatgttgatggaaaatgcttgggataagacacataaaaccaaacccccgggtgctgttggtgtgagcattgcacttgagaaattccttcctaataatccttcatctgacgttgggttgccaaggattgaaaatagcatcagtgtactagtctgttcaaagggtggcggtggactcttaaacgagcgcatggaacttgttgcagaactttgggaagctaacataaaggcccagtttgttcctcaggaagacccaagtcttcaagaacaatatgagtacgccagtgatcatgacatcaaatgccttgtatttatcactgaagcaggtctttcgcaaacagatcttgtgaaggttagacatctcgatgcaagaaaggagaaagatgttgaaaaggaagaaattgtgaagttcctgtctgaagcaatatctttacagttcaaaaatcctacaatctggagctgattggcaatctgctcgttagtgaattttgttcaggtttgctattccaaaggatcgatcggtatcagactttttatctacctgtcctggatataatctttctatggtcgggtgaaagcagtgttgcctttacaaacattttcacctacaccaaacgcattcatgatgctacagtacaagattgcaaggtgactggtggctttgtgcaatcttggttgtccctgcatgggccaagattatgcggtgtgtcttcagttcaagaaaagtgattgtcctggagcagtatcgcggtcgatgcaagctgacgggtagctgttgtacgacaatattggctgccaccattctgtggatagctagatggggagagttgcttctgcgatgtgactatggagctgcaatcctgcacaacgttcttgttgatagcggaacggctgtgcaacaggctcacctagttacgccctttgcaattttttggcttcaaattcttacagaaggcagtcttgtgcatagttcaaattgtttacaatcatgtacagaccaatttaacttctttgatgctcatagggaatgcccagtttgccagtgcagttttgctacatatcagtcagagtagagtgtatgtaacctgtgcacggtgctagagagcatcacttgattcaactcaagtgtttccgtcaaaaaaaaaaaaaaaaaaaaaa

SEQ ID NO: 2 - protein, 1247 aa, Triticum aestivum
MGHSARKKKKKGGAGRKAAKDHAAQLEGDQTALTDELTALAAIFLEDFKITSQSPHTRFSICIRPYSDGMGFGDLNVSAILDVICFAGYPHKCPKLRIIPEKNLCKEDADRLLSLLSDQANIYSREGRVMIFDLVEAAQEFLSEIAPATDSTSTAPHLGSSTIQEITDADVNASLDSGPYPGIFYIYNSFDLYGQLYEDNSWQRQGFDPTTDNARKNIGSQVNSNVRSKRKTVDEKSRFSADKINAAKSSSQDNAEHAMKHGVVREVVPSLPAVAEETDNDSKTLSTSNGGGMADTPERSFSSVHESEDSDLADDGWNDADSAPDSGSSNAPSHVSDMFDDASQNKKRDLILVHLLRLACASKDSLSAALPVISSELCNIGVLSEWAKQLISESPAVFGETFDHVFGQQMISSECSLFWRADNSSSRPNSRYLNDFEELRSLGQGGFGRVALCKNKLDGRQYAVKKIRLKDKSPQVNEKILREVATLSRLQHQHVVRYYQAWVETEYGQRHVVNTGGSRTAESSMYSFDEISLSDAGAGNKQESTYLYIQMEYCPRTLRQDFETYSSSFNVDHAWHLFRQIVEGLAHVHSQGIIHRDLTPSNIFFDVRNDIKIGDFGLAKFLKLEQLDHDQYIPTEGMGVSMDGTGQVGTYFYTAPEVEQKWPQINEKVDMYSAGVIFFELWHPFSTAMERHLVLTDLKQKGESPMSWSTQFPGQSNLLKRLLCPSPSERPSAIELLQNDLPPRMEDEWLNDVLRMIQTPEDTYVYDRVISTIFNEDRLVAKMQCQHESSKKSTCKNDNSELLDSIIEVSKEVFKRHCAKRFQISPLHTLDGKFTENSGQTVKILTQGGEMLELCYEQRTPFVMNVAANQLSSCKRYEISWVHRRAVGHSTPYRFLQGDFDIIGGSSPITQAEVIKVALDLVRRFYNSKAIVIRLNHSKLAEAVCSWAGVPQERRQNVAEFLSSTLVQYCPNKADRKSQWSLIRGQLLQDLRLSEEVVEKLHKADQRFCGSADLVLARLRGTLFYDKSACKALDDLSTFLKYLRIWSVEEHIAIDVLMPPSDYYYTDMFFQIYSKEGNPVLSSHEKLLAVGGRYDMLMENAWDKTHKTKPPGAVGVSIALEKFLPNNPSSDVGLPRIENSISVLVCSKGGGGLLNERMELVAELWEANIKAQFVPQEDPSLQEQYEYASDHDIKCLVFITEAGLSQTDLVKVRHLDARKEKDVEKEEIVKFLSEAISLQFKNPTIWS

SEQ ID NO: 3 - DNA, 24 bp, Artificial
  Synthetic nucleotide sequence
gccaatcagctccagattgtagga

SEQ ID NO: 4 - DNA, 26 bp, Artificial
  Synthetic primer sequence
atggggcacagcgcgaggaagaagaa

SEQ ID NO: 5 - DNA, 24 bp, Artificial
  Synthetic primer sequence
agtctgttcaaagggtggcggtgg

SEQ ID NO: 6 - DNA, 26 bp, Artificial
  Synthetic primer sequence
ggtggactcttaaacgagcgcatgga

SEQ ID NO: 7 - DNA, 20 bp, Artificial
  Synthetic primer sequence
accaataacacaggccgaag

SEQ ID NO: 8 - DNA, 19 bp, Artificial
  Synthetic primer sequence
caaggaccacgccgcgcag

SEQ ID NO: 9 - DNA, 26 bp, Artificial
  Synthetic primer sequence
gctaaatcgggtgtgaggtgattgtg

SEQ ID NO: 10 - DNA, 45 bp, Artificial
  Synthetic primer sequence
atttggccagtcggccgcggccgcgaagctttgagtggccgtaga

SEQ ID NO: 11 - DNA, 27 bp, Artificial
  Synthetic primer sequence
ccgctcgagcgggtgctcggtgttgtg

SEQ ID NO: 12 - DNA, 27 bp, Artificial
  Synthetic primer sequence
ccaaataaggcggatcgtaagtcacag

SEQ ID NO: 13 - DNA, 24 bp, Artificial
  Synthetic primer sequence
ccatggtcctgaaccttcacctcg

SEQ ID NO: 14 - DNA, 32 bp, Artificial
  Synthetic primer sequence
gcttgtaattaggcctatgtattcgaaccctc

SEQ ID NO: 15 - DNA, 25 bp, Artificial
  Synthetic primer sequence (AATx-02R)
agccatggccttcagctccagagtc

SEQ ID NO: 16 - DNA, 26 bp, Artificial
  Synthetic primer sequence (AATy-01F)
tcctgaacagtgggagaaactggcag

SEQ ID NO: 17 - DNA, 30 bp, Artificial
  Synthetic primer sequence (AATy-01R)
cagcctgacagaagatgcatcttcatcaag

SEQ ID NO: 18 - DNA, 28 bp, Artificial
  Synthetic primer sequence (AATz-01F)
atggcataccaaggatttgctagcggtg

SEQ ID NO: 19 - DNA, 25 bp, Artificial
  Synthetic primer sequence (AATz-01R)
ggatactcaggcatcccgctctctg

SEQ ID NO: 20 - DNA, 29 bp, Artificial
  Synthetic primer sequence (AKHSDH-01F)
agagatcgtctctgattcttgagaaggcg

SEQ ID NO: 21 - DNA, 24 bp, Artificial
  Synthetic primer sequence (AKHSDH-01R)
gcctcgctgtcattcaccaccaag

SEQ ID NO: 22 - DNA, 34 bp, Artificial
  Synthetic primer sequence (AKHSDH-02F)
gctactagtgaagtcagcatatcattgacactag

SEQ ID NO: 23 - DNA, 31 bp, Artificial
  Synthetic primer sequence (AKHSDH-02R)
gggaaatgattgatctgtgctgtaggagatg

SEQ ID NO: 24 - DNA, 32 bp, Artificial
  Synthetic primer sequence (AlaAT-02F)
ctgatgcattctatgctcttcgtctccttgag

SEQ ID NO: 25 - DNA, 27 bp, Artificial
  Synthetic primer sequence (AlaAT-02R)
gaacgtctcatggaacaccgtgaagcg

SEQ ID NO: 26 - DNA, 25 bp, Artificial
  Synthetic primer sequence (ALS-02F)
gcatcaggagcacgtgctgcctatg

SEQ ID NO: 27 - DNA, 31 bp, Artificial
  Synthetic primer sequence (ALS-02R)
attgcgcatgtcacacttgtaggtcttgtag

SEQ ID NO: 28 - DNA, 34 bp, Artificial
  Synthetic primer sequence (AS1-03F)
gataagatgatgtcaaatgcaaagttcatctacc

SEQ ID NO: 29 - DNA, 22 bp, Artificial
  Synthetic primer sequence (AS1-03R)
gccgtgctgcatgcaacgcttg

SEQ ID NO: 30 - DNA, 24 bp, Artificial
  Synthetic primer sequence (AS1-04F)
gagcagttcagtgatggtgttggc

SEQ ID NO: 31 - DNA, 22 bp, Artificial
  Synthetic primer sequence (AS1-04R)
gcaccgtcaggatcgccgagtt

SEQ ID NO: 32 - DNA, 28 bp, Artificial
  Synthetic primer sequence (CGS-01F)
catgtcctactgggattcgaaggagcag

SEQ ID NO: 33 - DNA, 27 bp, Artificial
  Synthetic primer sequence (CGS-01R)
caggtcctcaaaatcctgactccgatg

SEQ ID NO: 34 - DNA, 18 bp, Artificial
  Synthetic primer sequence (CyclophilinF)
caccgtcccctgcaattg

SEQ ID NO: 35 - DNA, 21 bp, Artificial
  Synthetic primer sequence (CyclophilinR)
agcccaccttctcgatgttct

SEQ ID NO: 36 - DNA, 20 bp, Artificial
  Synthetic primer sequence (DHDPS1-01F)
attgtcgaagccattggacg

SEQ ID NO: 37 - DNA, 24 bp, Artificial
  Sythetic primer sequence (DHDPS1-01R)
tattcaatacctgctgatcaacac

SEQ ID NO: 38 - DNA, 28 bp, Artificial
  Synthetic primer sequence (DHDPS1-03F)
catacactcccctccctcttgagaagag

SEQ ID NO: 39 - DNA, 33 bp, Artificial
  Synthetic primer sequence (DHDPS1-03R)
gctgatcaacacgaaatcatcgtcatccagaac

SEQ ID NO: 40 - DNA, 25 bp, Artificial
  Synthetic primer sequence (DHS2-02F)
gaccaagaaggaagccacccaggag

SEQ ID NO: 41 - DNA, 22 bp, Artificial
  Synthetic primer sequence (DHS2-02R)
gtcgcagtgggtgtggtagcgg

SEQ ID NO: 42 - DNA, 24 bp, Artificial
  Synthetic primer sequence (eIF2a-01F)
gagggcatgatcctcttctccgag

SEQ ID NO: 43 - DNA, 20 bp, Artificial
  Synthetic primer sequence (eIF2a-01R)
cccggcgcttggagaggtcg

SEQ ID NO: 44 - DNA, 29 bp, Artificial
  Synthetic primer sequence (GAPDH-F)
accccttcatcaccaccgactacatgacc

SEQ ID NO: 45 - DNA, 25 bp, Artificial
  Synthetic primer sequence (GAPDH-R)
ggatctcctcagggttcctgcagcc

SEQ ID NO: 46 - DNA, 27 bp, Artificial
  Synthetic primer sequence (GCN2-02F)
gcggatcgtaagtcacagtggagtttg

SEQ ID NO: 47 - DNA, 32 bp, Artificial
  Synthetic primer sequence (GCN2-02R)
ccctcttaatctagcaagtactagatctgcag

SEQ ID NO: 48 - DNA, 28 bp, Artificial
  Synthetic primer sequence (GCN2-03F)
ggctgctttgccagtaatatcatcggag

SEQ ID NO: 49 - DNA, 31 bp, Artificial
  Synthetic primer sequence (GCN2-03R)
agagagcactctgaagatatcatttgctgcc

SEQ ID NO: 50 - DNA, 24 bp, Artificial
  Synthetic primer sequence (HDH-01F)
cgccctcgcattgacttcacctcc

SEQ ID NO: 51 - DNA, 28 bp, Artificial
  Synthetic primer sequence (HDH-01R)
cacaacggcattatcgagagtgaccttg

SEQ ID NO: 52 - DNA, 23 bp, Artificial
  Synthetic primer sequence (NR-01F)
ggtactggtgctggtgcttctgg

SEQ ID NO: 53 - DNA, 28 bp, Artificial
  Synthetic primer sequence (NR-01R)
gaaccagcagttgttcatcatgcccatg

SEQ ID NO: 54 - DNA, 28 bp, Artificial
  Synthetic primer sequence (PAT1-01F)
gcgttcaacgcaaaagttctccaggatg

SEQ ID NO: 55 - DNA, 21 bp, Artificial
  Synthetic primer sequence (PAT1-01R)
cggagcgctgcgtctcctgtg

SEQ ID NO: 56 - DNA, 22 bp, Artificial
  Synthetic primer sequence (PP2A-02F)
cttgaagcgcctggcggaggag

SEQ ID NO: 57 - DNA, 29 bp, Artificial
  Synthetic primer sequence (PP2A-02R)
ggatggtcatgcgatacagataatgtggg

SEQ ID NO: 58 - DNA, 28 bp, Artificial
  Synthetic primer sequence (PSP-01F)
gttccatttgaagaggctcttgctgccc

SEQ ID NO: 59 - DNA, 25 bp, Artificial
  Synthetic primer sequence (PSP-01R)
cctgggtggcctcttctccaaacag

SEQ ID NO: 60 - DNA, 24 bp, Artificial
  Synthetic primer sequence (SDH-01F)
gagacgctccatttgctctccgtg

SEQ ID NO: 61 - DNA, 26 bp, Artificial
  Synthetic primer sequence (SDH-01R)
atcttctgtcgcagagctcctaaggg

SEQ ID NO: 62 - DNA, 21 bp, Artificial
  Synthetic primer sequence (SDI1ex1-01F)
ccgtatgcacgggccaagcac

SEQ ID NO: 63 - DNA, 23 bp, Artificial
  Synthetic primer sequence (SDI1ex2-01R)
gctgcttcatcaccaccgccatg

SEQ ID NO: 64 - DNA, 23 bp, Artificial
  Synthetic primer sequence (SDI1ex2-01F)
ggaagcagtcgcaggagtccctg

SEQ ID NO: 65 - DNA, 26 bp, Artificial
  Synthetic primer sequence (SDI1ex3-01R)
gcttgagcagctcgatctcctccttg

SEQ ID NO: 66 - DNA, 30 bp, Artificial
  Synthetic primer sequence (1433NR-02F)
ctctatggacatccgacaccaatgaggatg

SEQ ID NO: 67 - DNA, 30 bp, Artificial
  Synthetic primer sequence (1433NR-02R)
acctcactgtccgtctccagattcttttgg

SEQ ID NO: 68 - protein, 340 aa, Triticum aestivum
MPPNLECRMYEPRFPEVDAAVMIQVKHIADMGAYVSLLEYNNIEGMILFSELSRRRIRSISSLIKVGRQEPSMVLRVDRDKGYIDLSKRRVSEEEAATCEDRYNKSKLVHSIMRHAADTLGVDLEPLYQRIGWPLYRRYGHAFEAFKLIVTDPDAVLDVLTYEETVVGPDGEEVTKVVPAVTPEIKETLVQNIRRRMTPQPLKIRADIEMKCFQFDGVLHIKQAMKKAEAAGNDNCPVKIKLVAPPLYVLTTQTLDKDQGISVLTDAVKACTAEIEKYKGKLVVKEPPRAVSEREDKLFLDQIDSLMEQNAEVDGDVDSEEEEDTGMGNVDLTNSGVTAY

SEQ ID NO: 69 - protein, 19 aa, Unknown
  Absolutely conserved sequence across all organisms as diverse as yeast, humans and plants
NIEGMILFSELSRRRIRSI

SEQ ID NO: 70 - DNA, 763 bp, Artificial
  Wheat genome data contig 1723930
atttatcactgaagcaggtctttcgcaaacagatcttgtgaaggtaaatatgtgaggttatattaccttattgtgtttttacactgttaagaactggtgttttattatcatcgtcacaaaataaagaggctgtgacatgaataccagaatgatgtgcactgatttgcatttggaccatctgcttctattgtgagtacaaatcttaagaaaagctcaaatctggttttacagttgtgcctgtttttctttactgtaccttcttgctaggaagacttcttgagtgtcttgtatgaattcaacatattttcaaaaacattggatttctttctttattttggataatctttgagtttgctgggttccatctgaaaaatctgttacgtgatatgttttctgctgtcctttattctaatgtcgtacggaggcacgctaaaaacctcatgcaaaatagtgaaaattttaggatagatacaaacttgaccattgatgtctatttcgacgaggaaaattaacgtcaaggaaaaccgcggatcgttctacctgcacttggttcacaggaaattactaatgtttctaagcttttattatctttggttcaggttagacatctcgatgcaagaaaggagaaagatgttgaaaaggaagaaattgtgaagttcctgtctgaagcaatatctttacagttcaaaaatcctacaatctggagctgattggcaatctgctcgttagtgaattttgttcaggtttgctattccaaaggatc

SEQ ID NO: 71 - DNA, 2200 bp, Artificial
  Triticum Sp. sequence data
cagaacagaccatcagagagcccggttacaccatccagacagttgttagctcccgttgtccatttctatgccggggagtgggatgagctccccacaattctctcagttgtgcatccacctgcacaaccaacaaaccttagcttctctgaggatgattttatggatggctgcagtagccatgacggaagcagtatagttggtcaggaccccaggcgatcgaggaagctttctggcagccgtgcatgggagagggccagcgagaccgacccagcagatggagggtacgatgtaatttgatttctgagatccctaatgcggtgaactctctaaggaagctctatgcccttatcactaaggtcagttcctccatcatcaccttctcatgtgtcatatgttacctcgtgcatgtagccgatggagcacagtacgatttacttattccaagtatttaactgtgcatgttacgcttgcagtgcctacgccctccttatggagtcttgtatgtggcttcaaagaagaacttggttggctcaaatggcggcgcaaggcagcttaaaagtttgatggaggaggaaggtgtccttggtggtcacttcttgactgagatatctgaccgggagatatggaagttctttttcaagtaaaccggcaaatgttggactgaaaaagatgatcctggccatagtgtcgtatacaagaaaaattcatttgtctactcgcagcgctgtacataccgcgaagcttagttcgattaattctttggtgataggttcaagtatgcgtcccagttgttaaacctgcaaatgtgttggcattttaacccttttgtagatctcctatctcctaggtgactgttgagcttttgcataccgaatctatggtgctggcatatcacagcacgctatcagcaatatgtacaaatggaatggttactgtacttgcttgtcggtcttacttgaatcgtgtctgtcgtgtctttgttttgagagcttgttctttctttaaggttgaaatattttattataccagtagttcagatttaaggaagcacaatctcaacaaaggacttatcatgcgcatgcataattaagcgagggatctgttagatttgtttctctaaggaaatctgttagctacagtacaccataatgatttgagttcatgtcaagaacccgatcctctcatgaatgctttgagtttgtgtatcagtgtagtgtatgatagtgtgagtgttgcgtgtgtttgccttgaactcttgctacatgtcaacaaacaaatttggtaatatctaccctcacaaactttttactgcaatcactgccagtaatagaagagactttatttcaacacatcattgttgtcgccacctcaccgatgccacttagaaaatagaacctaaaaaagagacctattaagactggaattaacaaaaatgattattaacgaaggatccctttagttgctggggatggcatttgcaaaagtttttatggcaaattcttcaagcacacatggcaatttctttggaaacatatatcaaattctcgcaaacaacggatttgttgtgaaaattgccatgtcgcctgaaggaattgcctaactttcaacacataaattgccgcaaaaaacgttcattttgctgtgcccctagcgaatgttcgctgaataacattgtccttaacgaaaggatggatcccactcatctatctgctgacggagtctcgagaaagagaaggaggcggcggggtggaggagacactgggtggcggctagagcagaggaaggaaacgctcaaggtctcgccgcctctgcgctgactggatcgagatagcagtctggaccccggtctcgacgcacgcagcccttccagtccatcgctccattccacgtcaaaacctcctccccggactcgagtcagagaaaccccaacgcagatcccctcctcgtcagaccatcctccctcacccgaaacatccagtccgatcaatcccgtctccccgccggaccaaatcgccgccgccgcggccaccctcctctggtccggcgctgcctccgacgcggccgcgcgcgctccgctccgatggggcacagcgcgaggaagaagaagaagaagggcggcgccgggcgcaaggcggccaaggaccacgccgcgcagctcgagggcgaccagaccgcgctcacggacgagctcacggcgctg

SEQ ID NO: 72 - DNA, 120 bp, Artificial
  TaGCN2 PCR product
atggggcacagcgcgaggaagaagaagaagaagggcggcgccgggcgcaaggcggccaaggaccacgccgcgcagctcgagggcgaccagaccgcgctcacggacgagctcacggcgctg

SEQ ID NO: 73 - DNA, 1203 bp, Artificial
  Triticum Sp. eIF2 alpha nucleotide sequence is represented by SEQ ID NO 73 and 74.  The nucleotide sequence is divided due to the presence of an intron.
agaatttttttacgagaggagttagtgaccagatttgcgtataggctggtgaccaaggcgacacatgagaggtaattttagtttggacattgacaaattaaaatcaaacaaacatgacaaataaataggacaaaaaaacttagatatgagatatatctcacatctagatgtgacatagacggactctatttaggaatcaaatatctaatttcggtgtccgagcacggaggtaactcaaataaatcgggctgaaacctgtatccacacgaggatggacacgcatccccgtttggcgtcttcataaacggttcacgtactacacggcccagccacaccgtcgattcgcagctcgctcgccaatccaaccaatccataacccggctgcccctcgcccgtctccgccgcacccccaaaaccctagctcccctccccaccccaccccgtccccccaccgcctccggccgccgccgccgcccgcgaagatgccgccgaacctcgagtgccggatgtacgagccgcggttcccggaggtggacgcggcggtgatgatccaggtgaagcacatcgccgacatgggcgcctacgtctccctcctcgagtacaacaacatcgagggcatgatcctcttctccgagctctcccgccgccgcatccgctccatctcctccctcatcaaggtcggccgccaggagccctccatggtgctccgcgtcgaccgcgacaagggctacatcgacctctccaagcgccgcgtctccgaggaggaggccgccacctgcgaggaccgctacaacaagtccaagctcgtccactccatcatgcgccacgccgccgacaccctcggcgtcgacctcgagcccctctaccagcgcatcggctggccgctctaccgcaggtacggccacgccttcgaggccttcaagctcatcgtcaccgaccccgacgccgtgctcgacgtcctcacctacgaggagaccgtcgtcggccccgatggcgaggaggttcgttcccgaccccccttccccctccccctctccccaacggctaattaactgctcaagtattgggcgtactccaaacaataccatcaatttacttcgggcaggttgtttgttgtataacttgacactaatataccaaccgattagacctttacatgtgtttttcaagtaggtattccttagttagtagtactgcagtctg

SEQ ID NO: 74 - DNA, 556 bp, Artificial
  Triticum Sp. eIF2 alpha nucleotide sequence is represented by SEQ ID NO 73 and 74.  The nucleotide sequence is divided due to the presence of an intron.
tttagagtttttgccagaaaatgaaagtgatacttaaatttactggcaggtgactaaggtggtgcctgctgtcacccctgagattaaggagaccctggtccagaatatacggaggaggatgacaccgcagccactcaagatccgtgctgacatagagatgaaatgcttccaatttgatggagtgctccatattaagcaagctatgaagaaagctgaagctgcgggcaatgataattgtcctgtgaagattaaactagttgctcctccactttatgttctgaccacacagacccttgacaaggaccaagggatttcagttctcactgatgcagtcaaggcttgtacagcagagattgaaaaatacaaaggaaagttagtagtgaaggaaccaccaagagctgttagcgagcgggaagacaagctattcctcgaccagattgattccctaatggaacaaaacgcagaggtcgatggtgacgtggacagtgaagaggaggaagatacaggaatgggcaacgttgatcttacaaattctggcgtcactgcttactaaggc

(19) United States
(12) Patent Application Publication
(10) Pub. No.: US 2015/0176019 A1
(43) Pub. Date: Jun. 25, 2015

(54) Over-expression of GCN2-Type Protein Kinase in Plants

(71) Applicants: Nigel Halford, Harpenden (GB); Ed Byrne, Harpenden (GB); Ian Prosser, Harpenden (GB)

(72) Inventors: Nigel Halford, Harpenden (GB); Ed Byrne, Harpenden (GB); Ian Prosser, Harpenden (GB)

(21) Appl. No.: 14/344,798

(22) PCT Filed: Sep. 14, 2012
(86) PCT No.: PCT/GB2012/052280

§ 371 Date: Oct. 10, 2014

(30) Foreign Application Priority Data
Sep. 14, 2011 (GB) 1115893.8

(51) Int. Cl. C12N 15/82; C12N 9/12

(52) U.S. Cl. CPC C12N 15/8251; C12N 9/12; C12Y 207/11001

(57) ABSTRACT

Methods and compositions to achieve a significant reduction in free amino acid concentration in plants and a mitigation of the effects of sulphur deficiency by over-expression of GCN2.

### FIELD OF THE INVENTION

Methods and compositions to achieve a significant reduction in free asparagine concentration in plants, mitigation of the effects of sulphur deficiency, improving heat processability, yield and resistance to abiotic stress.

### BACKGROUND OF THE INVENTION

Interest in the control of free amino acid accumulation in cereal grain and other important crop products has been stimulated in recent years because free amino acid concentrations have been shown to affect processing properties and product quality. Free amino acids react with reducing sugars in the Maillard reaction, a complex series of non-enzymatic reactions that occurs during frying, baking, roasting and high-temperature processing. The products of the Maillard reaction include melanoidin pigments and complex mixtures of compounds that impart flavour and aroma (Mottram, 2007; Halford et al., 2011). However, the Maillard reaction also produces undesirable compounds and these include acrylamide, which was discovered in many popular foods in 2002 (Tareke et al., 2002). Acrylamide is formed if the amino acid that participates in the reaction's final stages is asparagine (Mottram et al., 2002; Stadler et al., 2002). Acrylamide is neurotoxic, carcinogenic and genotoxic in rodents and has been classified as a probable human carcinogen by the World Health Organisation (Friedman, 2003). The reduction of free amino acid and specifically free asparagine accumulation in, for example, wheat or other grain, maize, potatoes and other crops is therefore highly desirable. In wheat, sulphur deprivation has a particularly dramatic effect, causing increases of up to 30-fold in free asparagine concentration in the grain (Muttucumaru et al., 2006; Granvogl et al., 2007; Curtis et al., 2009).

Various attempts have been made in the art to address this need, including, for example, US20070074304, which reported a method for reducing the acrylamide content in a heat-processed plant product by reducing asparagine levels in the plant that is used to produce the product by expressing a gene that is involved in asparagine biosynthesis and a gene involved in asparagine metabolism.

Similarly, in EP 1 974 039, which disclosed a method for reducing the acrylamide content in a heat-processed plant product which involved reducing asparagine levels in the plant that is used to produce the product by expressing in the plant a polynucleotide that has the complete or partial sense or antisense sequence of the coding or non-coding sequence of a gene that encodes an enzyme that catalyses the synthesis of asparagine from aspartate.

Free amino acid concentration is regulated and co-ordinated with protein synthesis in yeast (Saccharomyces cereviseae) by a regulatory protein kinase, general control nonderepressible-2 (GCN2). The name arises from the fact that general control of amino acid metabolism is in a permanently repressed state in gcn2- and other gcn-mutants. In U.S. Pat. No. 6,677,502, it was stated that:

“a member of the ATP-binding cassette (ABC)-superfamily, GCN20, uptakes ions and amino acids in yeast. GCN20 is co-immunoprecipitated from cell extracts with GCN1, another factor required to activate the regulatory protein kinase, GCN2, and the two proteins interact in the yeast two-hybrid system. These two factors indicate that GCN1 and GCN20 are components of a protein complex that couples the kinase activity of GCN2 to the availability of amino acids. GCN20 is closely related to ABC proteins identified in Caenorhabditis elegans, rice and humans, suggesting that the function of GCN20 may be conserved among diverse eukaryotic organisms (Vazquez de Aldana, C. R. et al. (1995) EMBO J 14:3184-3199). As part of the GCN1/GCN20 complex, GCN20 may be involved in the modulation of the EF3-related function which facilitates the activation of GCN2 by uncharged tRNA on translating ribosomes (Marton, M. J. et al. (1997) Mol Cell Biol 17:4474-4489).”

The primary focus of the disclosure in U.S. Pat. No. 6,677,502 is GCN20 in yeast and the effect it has on GCN2 function in yeast. There is nothing in it that covers or predicts the use of GCN2 manipulation in a plant to reduce acrylamide forming potential. It is unclear if GCN20 has been identified in plants.

In U.S. Pat. No. 6,692,962, it was stated that:

“Plant eIF2α Complements Yeast eIF2α Under GCN4 Derepressing Conditions. Note that eIF2α (eukaryotic translation initiation factor 2α) is the substrate for GCN2, while GCN4 is a transcription factor that is upregulated translationally when eIF2α is phosphorylated by GCN2. Wild type wheat and yeast eIF2α proteins are specifically phosphorylated in vitro on serine 51 by yeast GCN2. However, Krishna V M, Janaki N and Ramaiah K V A: Arch Biochem Biophys 346: 28-36 (1997), found that even though wheat germ eIF2α was phosphorylated in vitro, it did not mediate translational initiation in reticulocyte lysates; thus, the in vivo significance of phosphorylation remains unclear. In order to address this issue in vivo, yeast strains expressing wheat eIF2α proteins were grown under conditions that induce activity of the endogenous yeast eIF2α kinase, GCN2. These conditions were created by the addition of 3-aminotriazole (3-AT), an inhibitor of histidine biosynthesis. Previous studies established that resistance to 3-AT requires an intact eIF2α phosphorylation pathway. Strains expressing wild type plant eIF2α were 3-AT resistant after 3d incubation. No significant difference was apparent between the growth of strains expressing wild type plant or yeast eIF2α. However, the ability to grow under nutrient starvation conditions was conferred by serine 51S of eIF2α and, by extension, phosphorylation, because expression of a non-phosphorylatable mutant, 51A, of plant or yeast eIF2α, with alanine at position 51 in place of serine, inhibited strain growth under these conditions. During the course of this study it was noted that growth of strains expressing yeast 51A remained suppressed even after long term incubation while partial growth was observed in strains expressing plant 51A after 4d incubation.

Growth under nutrient starvation conditions is mediated by ternary complex formation that is conditioned not only by eIF2α phosphorylation but also by activity of the eIF2 holoenzyme. The only eIF2α kinase in yeast is GCN2. Thus, it was important to evaluate the contribution of GCN2 activity. Isogenic gcn2-strains were therefore transformed with plant and yeast 51S and 51A constructs and following selection on 5-FOA, strains were plated on media in the presence and absence of 3-AT. The absence of GCN2 had no significant effect on strain growth under nutrient rich conditions. However, after 3 days incubation on media containing 30 mM 3-AT, no growth was observed in gcn2-strains expressing plant or yeast eIF2α 51S or 51A. After 4d, as previously observed, strains expressing plant constructs showed slight growth relative to strains expressing yeast 51S or 51A, suggesting a partial GCN2 independent growth effect.

The GCN2 dependent growth response under nutrient starvation conditions was further evaluated in strains that constitutively express GCN2. Constitutive expression of GCN2 suppresses growth of strains expressing yeast eIF2α 51S under nutrient rich conditions due to decreased ternary complex formation resulting in a general decrease in protein synthesis. However, under starvation conditions yeast 51A-expressing strains are unable to grow whereas 51S strains grow, albeit less than in a GCN2 background. Thus, the functional substitution of plant eIF2α would predict that strains expressing plant eIF2α 51S in a GCN2c background would show growth suppression under non-starvation but not under starvation conditions relative to 51A expressing strains. To test this prediction, strains containing plant eIF2α proteins were transformed with the GCN2c-517 allele; that is a dominant mutation resulting in high constitutive expression of GCN2. Growth of the 51S-expressing strain was suppressed on nutrient rich medium while the 51A strain was unaffected by the GCN2c-517 allele. However, under nutrient deprivation conditions only the 51S strain was able to grow. Consistent with previous data, the 51A strain grew slightly on 3-AT medium following 4d incubation.

Plant eIF2α is specifically phosphorylated on Serine 51 by GCN2. In vivo plant eIF2α phosphorylation levels in the various strain backgrounds were directly determined under GCN4 repressing (non-starvation) and derepressing (starvation) conditions by isoelectric focusing and immunoblotting. Under GCN4 repressing conditions only a basic band was observed in GCN2 strains expressing either 51A or 51S, indicating the presence of the unphosphorylated species (lanes 1, 3). No phosphorylated acidic band was detected under the isoelectric focusing conditions used or in immunoblotting experiments using antiserum that specifically recognizes the phosphorylated form of wheat eIF2α. This is in slight contrast to the results of Dever et al. who found that yeast eIF2α is normally present under nutrient rich conditions as phosphorylated and nonphosphorylated species but is hyperphosphorylated under GCN4 derepressing conditions. An additional more acidic band and a band corresponding to phosphorylated eIF2α was observed under starvation (GCN4 derepressing) conditions in GCN2 containing strains expressing plant 51S but not 51A. In the absence of GCN2, regardless of growth conditions, plant eIF2α was not phosphorylated (lanes 5-8). Further, in GCN2c-51S but not 51A strains eIF2α was phosphorylated, as expected, under GCN4 repressing and derepressing conditions, although phosphorylation levels increased under GCN4 derepressing conditions. These data confirm the specific in vivo GCN2-dependent phosphorylation of plant eIF2α and link phosphorylation with the ability of strains to grow under starvation conditions that require an intact general amino acid control pathway.

Phosphorylation of eIF2α Induces Expression of GCN4

GCN4 expression is an extremely sensitive indicator of ternary complex activity and thus provides a direct method to measure the impact of eIF2α phosphorylation on translation. Isogenic strains expressing wheat 51S or 51A contained a GCN4-lacZ fusion allowing measurement of β-galactosidase activity as a function of GCN4 expression level. Under starvation conditions GCN4 is expressed early prior to any phenotypic response. Table 1 shows that β-galactosidase activity dramatically increased in plant 51S-expressing strains under nutrient starvation conditions relative to non-starvation conditions and that activity was GCN2 dependent. The GCN2 dependent nature of this response was supported by β-galactosidase measurements from gcn2 and GCN2c strains. In the absence of GCN2, there were no significant differences between GCN4 expression level under derepressing or repressing conditions regardless of eIF2α species. Constitutive expression of GCN2 in 51S-containing strains caused a significant increase in β-galactosidase activity under non-starvation conditions relative to isogenic strains carrying GCN2. The 51A mutation that inhibits growth under amino acid starvation conditions also suppressed GCN4 expression relative to strains expressing plant 51S. These data are consistent with the functional substitution of wheat eIF2α in the yeast phosphorylation-mediated translational control pathway.

In vivo Regulation of Protein Synthesis by Phosphorylation of the α Subunit of Wheat Eukaryotic Initiation Factor 2.”

This disclosure in U.S. Pat. No. 6,692,962 refers thus to the use of a modified form of eIF2alpha, the substrate for GCN2. It predates the discovery of acrylamide in plant-derived foods. It claims a use for modified eIF2alpha in pathogen defence, based on Roth's assertion that a PKR-like activity was present in plants. PKR is a mammalian protein kinase that is related to GCN2 and phosphorylates the same substrate but in response to virus infection rather than amino acid deficiency. The patent also predates the publication of the Arabidopsis genome, when it became clear that plants do not have a PKR homologue (the GCN2 homologue is the only eIF2alpha kinase in plants). The patent neither discloses nor suggests asparagine synthetase, or sulphur signalling/responses, or acrylamide formation.

To the best of our knowledge, there is no specific report which links GCN2 manipulation with reduction in, specifically, Asn. Accordingly, prior to the present patent disclosure, there has been no basis to predict that the acrylamide problem could potentially be ameliorated by modification of GCN2 activity. Nor could it have been predicted whether increasing or decreasing GCN2 activity would result in increases or decreases in Asn concentration in plant tissue. Over-expression of GCN2 might have been expected to cause a general increase in free amino acid levels, based on work in yeast. In fact, as we show herein, the opposite occurs, and there are different effects on different genes involved in amino acid biosynthesis. Clearly, the plant system is much more complicated than the general amino acid control system of yeast, and is not yet fully understood. The amino acid and sulphur responses we report herein are completely unexpected and could not be predicted from the results of studies in other organisms.

By contrast, as will be apparent from a review of the entire disclosure, the present invention provides a method of modifying free amino acid concentration and/or sulphur signalling in plants by over-expression (decrease free AA) or silencing (increase free AA) of GCN2; resulting in crops that have more favourable heat processing qualities. In addition, the plants also exhibit evidence of greater yield, resistance to abiotic stress, including, but not limited to, nutritional stress and better nitrogen utilization (nitrate reductase gene expression is affected).

The present patent disclosure takes a very different approach, as compared with the approach taken in the art, (see, for example, the above-discussed U.S. Pat. Nos. 6,692,962 and 6,677,502) to achieve a similar goal. To fully appreciate the contribution made by the present invention, and, indeed, to fully appreciate the invention, it is necessary to provide some background on certain aspects of plant biochemistry.

Translation initiation, the point at which a ribosome recruits an mRNA molecule, is a key control point for protein synthesis in all eukaryotic species. It is regulated by phosphorylation of the a subunit of eukaryotic translation initiation factor 2 (eIF2α) (reviewed by Hershey and Merrick, 2000). eIF2 is a trimeric factor (subunits α, β and γ) that can bind either guanosine diphosphate (GDP) or triphosphate (GTP). Only when bound to GTP is it able to carry out its physiological function of binding Met-tRNA to the ribosome and transferring it to the 40S ribosomal subunit. Following attachment of the [eIF2.GTP.Met-tRNA] complex to the 40S subunit, the GTP is hydrolysed to GDP. Phosphorylation of eIF2α inhibits the conversion of eIF2-GDP to eIF2-GTP, preventing further cycles of translation initiation and suppressing protein synthesis (Wek et al., 2006).

In budding yeast (Saccharomyces cerevisiae), phosphorylation of eIF2α not only causes a general reduction in protein synthesis, but also initiates a change in expression of a large number of genes, most notably involved in amino acid biosynthesis. Thus, under conditions of amino acid starvation, yeast is able to switch on amino acid biosynthesis genes, helping the cell to maintain homeostasis and survive. This ‘general amino acid control’ is orchestrated by the transcription factor GCN4 (General Control Non-derepressible 4) (Hinnebusch, 1997; 2005), the name arising from the fact that general amino acid control is in an irreversibly repressed state in gcn4 and other gcn mutants. In budding yeast, GCN4 levels are regulated post-transcriptionally, the synthesis of GCN4 increasing when eIF2α is phosphorylated due to translation proceeding from an initiation codon that is not used under normal conditions (Hinnebusch, 1992; 1994). GCN4 promotes the expression of genes encoding enzymes in every amino acid biosynthetic pathway except cysteine, as well as many other genes involved in a wide range of cellular processes (Natarajan, 2001). In mammals, phosphorylation of eIF2α leads to an increase in translation of ATF4, the functional orthologue of GCN4. Increased levels of ATF4 lead to induction of additional bZIP transcription regulators, ATF3 and CHOP/GADD153 (Harding et al., 2000).

The protein kinase that phosphorylates eIF2α was given the name GCN2 (Wek et al., 1989). In yeast, GCN2 is a relatively large protein kinase (1659 amino acid residues; 190 kDa) that senses a reduction in cellular amino acid content through the interaction of its regulatory domain with uncharged tRNA, the cellular concentration of which increases under conditions of amino acid starvation (Wek et al., 1989; 2003; Zhu et al., 1996). The GCN2 regulatory domain has some amino acid sequence similarity with Histidyl-tRNA synthetases and is sometimes called the Histidyl-tRNA synthetase-like domain. Activation involves a conformational change in GCN2 and autophosphorylation at two threonine residues in the conserved activation loop of the kinase domain. GCN2 may also be activated and protein synthesis inhibited in response to purine deprivation, exposure to UV-B light, oxidative and osmotic stress, or glucose deprivation (Hinnebusch, 2005; Mascarenhas et al., 2008; Yang et al., 2000).

Three other animal protein kinases are known to be able to phosphorylate eIF2α: double-stranded RNA-dependent protein kinase (PKR), PKR-like endoplasmic reticulum kinase (PERK) and haem-regulated inhibitor (HRI) (Nanduri et al., 2000; Chen and London, 1995; Kaufman, 1999). The four eIF2α kinases share a highly conserved protein kinase domain but their regulatory domains differ, enabling each kinase to respond to a different stimulus.

The first plant GCN2 homologue to be identified was AtGCN2 from Arabidopsis (Arabidopsis thaliana) (Zhang et al., 2003). The ATGCN2 protein is structurally similar to GCN2 from fungi and animals, with a characteristic eIF2α kinase domain adjacent to a Histidyl-tRNA synthetase-like regulatory domain, and it complements the gcn2 mutation of yeast (Zhang et al., 2003). However, it is smaller than yeast GCN2 (1241 amino acid residues; 140 kDa). Arabidopsis mutants lacking AtGCN2 grow normally in compost but are more sensitive than wild-type to herbicides such as glyphosate and chlorsulphuron that interfere with amino acid biosynthesis, an effect that can be reversed by feeding the plants with the appropriate amino acids (Zhang et al., 2008). These herbicides induce phosphorylation of eIF2α in wild-type Arabidopsis but not in gcn2 mutants (Zhang et al., 2008). GCN2-like ESTs and genomic sequences have since been identified in a variety of plant species (Halford, 2006), but have not been characterised in any detail. In all the plant species where full genome data is available, GCN2 is encoded by a single gene and is the only eIF2α kinase.

As in fungal systems, Arabidopsis GCN2 (AtGCN2) may be activated in response to other stress stimuli, such as purine deprivation, UV light, cold shock and wounding (Lageix et al., 2008). AtGCN2 is also activated in response to treatment with methyl jasmonate or salicylic acid, which are involved in the activation of defence mechanisms in response to insect herbivores, and aminocyclopropane carboxylic acid (ACC), which is involved in ethylene biosynthesis and therefore ripening and senescence (Lageix et al., 2008).

The discovery of a plant GCN2 homologue was evidence that a general amino acid control system, similar to that of fungi and animals, might exist in plants, at least in part. Previous studies had suggested that this might be so. For example, blocking histidine biosynthesis in Arabidopsis with a specific inhibitor, IRL 1803, had been shown to increase expression of eight genes involved not only in the synthesis of histidine but also the aromatic amino acids (tyrosine, tryptophan and phenylalanine), lysine and purines (Guyer et al., 1995). Genes encoding tryptophan biosynthesis pathway enzymes had also been shown to be induced by amino acid starvation caused by glyphosate application and other treatments in Arabidopsis (Zhao et al., 1998). In another study, the contents of most minor amino acids had been shown to vary in concert in wheat, barley and potato leaves (Noctor et al., 2000). However, although Zhang et al. (2008) showed that the expression of key genes of amino acid biosynthesis was affected by treatment of Arabidopsis with herbicides that affected amino acid metabolism, this response was also seen in mutants lacking AtGCN2 (Zhang et al., 2008). The only exception was a nitrate reductase gene, NIA1, the expression of which was reduced in the mutant plants. Furthermore, no obvious candidate for a GCN4 homologue is identifiable in plants based on amino acid sequence similarity (Halford, 2006).

Wheat GCN2 (TaGCN2) has not been characterised previously. However, wheat eIF2α has been reported to contain a conserved GCN2 phosphorylation site, although the full amino acid sequence of eIF2α has not previously been described. Yeast GCN2 has been shown to phosphorylate wheat eIF2α in vitro at this site (Chang et al., 1999) and wheat eIF2α complements eIF2α deletion mutants of yeast, restoring a fully functional general amino acid control system (Chang et al., 2000). In this patent disclosure, a polymerase chain reaction (PCR) product derived from the transcript of a GCN2-related gene (TaGCN2) was amplified from wheat leaf RNA and transgenic wheat plants were produced in which TaGCN2 was over-expressed. Analysis of these plants showed dramatic effects on free amino acid levels and gene expression and placed TaGCN2 irrefutably in the sulphur signalling pathway. We show that manipulation of TaGCN2 gene expression can be used to reduce free asparagine accumulation in wheat grain and the risk of acrylamide formation in wheat and other plant products.

### SUMMARY OF THE INVENTION

Over-expression of GCN2 enables the control of free amino acid concentration to improve nutritional safety in baking and frying of a range of crops, such as wheat, corn and potatoes, and confers a possible yield benefit.

The protein kinase GCN2 (General Control Nonderepressible 2) represents a key control factor for protein synthesis in all eukaryotic species. Without wishing to be bound by mechanistic considerations, it is known that GCN2 is activated in response to low free amino acid concentrations and acts by phosphorylation of the a subunit of eukaryotic translation initiation factor 2 (eIF2α), thereby reducing the rate of protein synthesis. Paradoxically, phosphorylation of eIF2α also leads to an increase in translation of a transcription factor, GCN4, resulting in the induction of expression of hundreds of genes, including many that encode enzymes involved in amino acid biosynthesis. Amino acid biosynthesis is therefore regulated in response to free amino acid concentrations and is co-ordinated with the control of protein synthesis. Other mechanisms may be at work as well, particularly in plants, in which the system is clearly more complicated than that of fungi and has been less extensively studied.

The control of free amino acid accumulation is an important aspect of crop quality because free amino acids react with sugars during heat processing (such as frying, baking and roasting) in the Maillard reaction. This reaction is important for colour, flavour and aroma development but also produces carcinogenic compounds; these include acrylamide, which forms when the amino acid taking part in the final stages of the reaction is asparagine. This is particularly accentuated in some crop species, notably wheat, in sulphur-deficient growing conditions, which cause the accumulation of high concentrations of asparagine in the grain. Therefore the ability to control free amino acid and particularly free asparagine levels helps to reduce the formation of dangerous chemicals in the processing of crops, especially wheat, corn, rye, other cereals and potatoes.

In an embodiment according to this invention, a GCN2 homologue (TaGCN2) from wheat has been cloned (having an open reading frame encoding a protein with 52% amino acid sequence identity with the Arabidopsis GCN2 (AtGCN2), 84% identity with an uncharacterised rice GCN2-type protein kinase, and the eIF2α kinase and adjacent Histadyl-tRNA synthetase-like regulatory domains typical of GCN2-type protein kinases (FIG. 1)). TaGCN2 was over-expressed in wheat under the control of an actin gene promoter. Of course, as those skilled in the art will appreciate, other promoters may be used, including, for example, cereal endosperm specific promoters, such as the Glu-1D-1 (HMW-1DX5) gene promoter of wheat (Lamachia et al., 2001), or potato tuber-specific promoters, such as patatin gene promoters (Rocha-Sosa et al., 1989). This affected expression of a number of genes as well as the amino acid profile of the resulting transgenic plants. Three independent homozygous lines, 395, 402 and 426, with a range of GCN2 expression levels (approximately 140%, 220% and 270%, respectively, compared with controls)(FIG. 2), were selected for detailed characterisation. Expression of translation initiation factor-2α (eIF2α), protein phosphatase-2A (PP2A) and nitrate reductase (NR) were each significantly increased in these transgenic lines (FIG. 3A).

Increases in expression of eIF2α and PP2A may represent plant responses to GCN2 over-expression because eIF2α is the substrate for GCN2 while PP2A is a protein phosphatase that reverses the phosphorylation of eIF2α by GCN2.

This patent disclosure reports that over-expression of GCN2 controls changes in a range of enzymes involved in amino acid biosynthesis and amino acid metabolism. It also indicates that GCN2 over-expression impairs plant responses to sulphur deficiency, including an undesirable increase in asparagine synthetase gene expression. Measurements of free amino acid concentrations in the seeds of homozygous T3 plants revealed that total free amino acid levels were reduced in the grains of all three transgenic lines (FIG. 4). In particular, free asparagine concentration was much lower than in controls (up to 70% reduction).

For a further comparison, transgenic plants in which TaGCN2 gene expression was inhibited by RNA interference (RNAi) were also produced and compared with controls and the TaGCN2 over-expressing lines. Expression of the RNAi construct was targeted to the seed endosperm with a Glu-1D-1 (HMW-1DX5) gene promoter from wheat (Lamachia et al., 2001). Five lines, 122, 138, 140, 208 and 215, showing a reduction in TaGCN2 gene expression, were identified (FIG. 5). Note that line 138 originated from the same transgenic event as line 140, while 208 originated from the same event as 215, so three independent transgenic events were represented in the five lines. The lines showed dramatic changes in free amino acid levels (FIG. 6). Compared with the controls, RNAi lines 122 and 208+215, and all three of the over-expressing lines, showed significant changes (p<0.05) in asparagine concentration (Tables 1 and 2). In over-expressing line 426, free asparagine concentration was 0.955 mmol kg⁻¹, compared with an average of 3.30 mmol kg⁻¹ in the controls, a reduction of more than 70%. The RNAi lines therefore confirmed the results obtained with the over-expressors, with reduced GCN2 resulting in much higher free amino acid levels, especially free asparagine levels.

Measurements of the grain yield and 1000 grain weight of the TaGCN2 over-expressing lines showed a trend for the over-expressing lines to yield more grain weight per plant than the control plants, although 1000 grain weight was similar in control and transgenic lines, indicating that there was no difference in the size of individual grains. The nitrogen content of the grain from the transgenic lines was lower than that of controls, while the carbon content was unchanged, meaning that the transgenic lines had a higher ratio of carbon to nitrogen than the controls. This effect is likely to be increased by abiotic stress factors.

In summary, the present invention disclosure demonstrates that RNAi silencing of GCN2 gives rise to significant increases in total free amino acid concentration in seeds of the RNAi plants, with free Asn and Gln being especially high. By contrast, in grain of plants over-expressing GCN2 there is a significant decrease in free amino acid concentration. GCN2 over-expression represses expression of Asn synthase (AS1), cystathione gamma-synthase and sulphur deficiency induced-1 (SDI1) genes, while genes encoding eIF2α, PP2A, nitrate reductase, phosphoserine phosphatase and dihydropicolinate synthase all increase in expression. In wild type plants deprived of sulphur, SDI1 and AS1 gene expression increase, but not in plants over-expressing GCN2, while in the latter, expression of genes encoding Asp kinase/homoserine dehydrogenase and 3-deoxy-D-arabino-heptulsonate-7-phosphate synthase is lower than in controls under S deficiency.

Accordingly, it is an object of this invention to provide an isolated GCN2 homologue from wheat.

It is another object of this invention to provide a plant over-expressing GCN2.

A further object of this invention is to provide a plant with reduced free amino acid levels.

A further object of this invention is to provide a plant with reduced asparagine levels in grain.

A further object of this invention is to provide a method for reducing asparagine in the grain of a plant.

A further object of this invention is to provide a method for using GCN2 to produce a plant with reduced asparagine.

A further object of this invention is to provide a method to inhibit a plant's response to low sulphur or sulphur starvation.

A further object of this invention is to provide grain of improved quality by reducing the formation of carcinogenic chemicals during heat processing.

A further object of this invention is to provide crop plants with increased yield of seed, grain, tubers or other harvested organs.

A further object of this invention is to provide plants with increased abiotic stress resistance.

Further objects and advantages of this invention will be appreciated from a review of the entire disclosure and appended claims.

### BRIEF DESCRIPTION OF THE DRAWINGS

FIG. 1 provides a Schematic diagram representing the structure of wheat GCN2-related protein kinase, TaGCN2. The relative positions of the GCN1 binding domain, eIF2α kinase domain, and regulatory domain (including anticodon binding sub-domain) are shown.

FIG. 2 shows relative expression of TaGCN2 in leaves of transgenic wheat lines in which TaGCN2 was over-expressed under the control of a rice actin gene promoter, compared with controls. The analysis was carried out in two separate quantitative real-time PCR experiments using different reference genes. In each case, expression in the control lines is represented as 1. Error bars represent standard error of the mean from analyses of two biological replicates.

FIGS. 3A and 3B show expression (normalised relative quantities (NRQ)) values of a suite of genes (Table 3) in leaves of transgenic wheat lines in which TaGCN2 was over-expressed under the control of an actin gene promoter, and in controls. The plants were grown with sulphur either supplied (S+) or withheld (S−). The analysis was carried out by quantitative real-time PCR using 3-phosphate dehydrogenase (GAPDH) and succinate dehydrogenase (SDH) as reference genes. Note that the graphs have different scales on the y-axis. In all cases shown, the levels of expression of the gene differed significantly (p<0.05) between the transgenic and control lines in either S+ or S− conditions, or in both. Full statistical analysis, including SEDs for making the appropriate comparisons of either lines, sulphur conditions, or all combinations of both these factors from ANOVA results, is given in Example 5. In the plants that were supplied with sulphur there were higher expression levels of phosphoserine phosphatase and dihydropicolinate synthase, while expression of an asparagine synthetase (AS) gene and cystathionine γ-synthase (CGS) and sulphur-deficiency-induced-1 (SDI1) were all significantly lower.

FIG. 4 shows concentrations (mmol kg⁻¹) of total free amino acids (left) and free asparagine (right) in the grain of transgenic wheat lines in which the expression of TaGCN2 was increased by constitutive over-expression, compared with controls: “Control A” for line 395 and “Control B” for lines 402 and 426. For statistical analyses, including the SEDs for making comparisons of the lines, refer to Table 1.

FIG. 5 shows relative expression of TaGCN2 in grain of transgenic wheat lines in which TaGCN2 gene expression was reduced endosperm-specifically by RNA interference, compared with wild-type and null segregant controls. Expression levels were determined by quantitative real-time PCR and expression in the wild-type control is represented as 1. Note that lines 138 and 140 arose from the same transgenic event, as did 208 and 215. Only technical replicates were performed so standard errors are not presented as they would not substantiate biological variation.

FIG. 6 shows free amino acid concentration (left) and free asparagine concentration (right) in the grain of transgenic wheat lines 122, 138, 140, 208 and 215, in which the expression of TaGCN2 in the seed endosperm was inhibited by RNA interference, compared with controls. For statistical analyses, including the SEDs for making comparisons of the lines, refer to Table 2.

FIG. 7 shows the TaGCN2 nucleotide sequence.

FIG. 8 shows the full nucleotide sequence of wheat genome data contig 1723930 (SEQ ID NO:70) with intron shown in bold.

FIG. 9 provides an alignment of TaGCN2 PCR product (SEQ ID NO:71) with wheat genome data (SEQ ID NO:72) at 5′ end, with nucleotides numbered with respect to the ATG translation start codon.

FIG. 10 provides the derived amino acid sequence of TaGCN2: 1247 residues; 140 kDa (SEQ ID NO:10).

FIG. 11 provides the wheat eIF2α nucleotide sequence (SEQ ID NOS: 73 and 24), with putative coding regions shown in bold and numbering referring to the coding region, beginning with the putative translation start site.

FIG. 12 provides the wheat eIF2α derived amino acid sequence of 340 residues (SEQ ID NO:68), and mass of 38.4 kDa; the serine residue that is phosphorylated by GCN2 is highlighted in black, and the surrounding residues that are conserved in all eIF2α sequences to date are highlighted in grey.

### DETAILED DISCLOSURE OF THE PREFERRED EMBODIMENTS ACCORDING TO THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature.

A key point of regulation of protein synthesis and amino acid homeostasis in eukaryotes is phosphorylation of the a subunit of eukaryotic translation initiation factor 2 (eIF2α) by protein kinase General Control Non-derepressible (GCN)-2. We disclose herein a GCN2-type PCR product (TaGCN2), amplified from wheat (Triticum aestivum) RNA, while a wheat eIF2α homologue was identified in wheat genome data and found to contain a conserved target site for phosphorylation by GCN2. TaGCN2 over-expression in transgenic wheat resulted in significant decreases in total free amino acid concentration in the grain, with free asparagine and glutamic acid concentration being much lower than in controls. There were significant increases in expression of eIF2α and protein phosphatase PP2A, as well as a nitrate reductase gene and genes encoding phosphoserine phosphatase and dihydropicolinate synthase, while expression of an asparagine synthetase (AS1) gene and genes encoding cystathionine gamma-synthase and sulphur-deficiency-induced-1 all decreased significantly.

Sulphur deficiency-induced activation of these genes occurred in wild-type plants but not in TaGCN2 over-expressing lines. Under sulphur deprivation, the expression of genes encoding aspartate kinase/homoserine dehydrogenase and 3-deoxy-D-arabino-heptulosonate-7-phosphate synthase was also lower than in controls. The examples and results disclosed herein demonstrate that TaGCN2 plays an important role in the regulation of genes encoding enzymes of amino acid biosynthesis in wheat and is the first to implicate GCN2-type protein kinases so clearly in sulphur signalling in any organism. It shows that manipulation of TaGCN2 gene expression could be used to reduce free asparagine accumulation in wheat grain and the risk of acrylamide formation in wheat products.

We show that over-expression of TaGCN2, the wheat homologue of AtGCN2, has profound effects on free amino acid concentrations in wheat grain and on the expression of several genes encoding key enzymes in amino acid biosynthesis. Free amino acid concentrations in the grain of the transgenic lines were decreased, mainly as a result of substantial reductions in the concentrations of free asparagine and glutamic acid. In one line, free asparagine concentration was reduced by more than two thirds compared with controls. Accordingly, based on this discovery, and depending on expression level, affected, for example, by codon choices, promoter and other factors known to those skilled in the art, it is clear that by routine experimentation, any desired level of decrease in free Asn may be obtained, including, but not limited to, 10%, 20%, 30%, 40%, 50%, 60%, 70% or greater reductions. There was some evidence that TaGCN2 over-expression could increase grain yield.

The data presented herein clearly shows TaGCN2 to be involved in the regulation of gene expression under normal conditions, and also implicates TaGCN2 in sulphur signalling. This is demonstrated dramatically herein in the analysis of asparagine synthetase (AS1) gene expression, which we show rose almost ten-fold in response to sulphur deprivation in wild-type plants but which was almost undetectable, with or without sulphur, in the transgenic lines. AS1 gene expression has been shown to be induced by salinity and osmotic stress (Wang et al., 2005) but has not previously been reported to increase in response to sulphur deprivation, although the fact that it does is not unexpected given the massive accumulation of asparagine seen in grain from sulphur-deprived wheat (Muttucumaru et al., 2006; Granvogl et al., 2007; Curtis et al., 2009). TaGCN2 over-expression also had profound effects on the expression of a gene used as a marker for sulphur deficiency, sulphur deficiency inducible-1 (SDI1), and a gene encoding cystathionine gamma-synthase (CGS). Expression of these genes was significantly reduced in the transgenic plants compared with controls when the plants were supplied with sulphur and SDI1 also showed no induction in the transgenic lines in response to sulphur deficiency, whereas its expression increased significantly in controls.

The involvement of GCN2 or a related protein kinase in sulphur signalling has not been demonstrated so clearly in any organism before. However, phosphorylation of eIF2α, the substrate for GCN2, has been shown to be higher in liver cells of rats fed a diet deficient in sulphur-containing amino acids than in well-nourished rats (Sikalidis and Stipanuk, 2010). Fascinatingly, that study showed that asparagine synthetase gene expression was also increased.

The discovery of acrylamide in many popular foods (Tareke et al., 2002) has stimulated great interest in the control of free amino acid and particularly free asparagine accumulation in grains, tubers and other crop products. Acrylamide forms as part of the Maillard reaction, a series of non-enzymatic reactions between reducing sugars and amino groups, principally those of amino acids. The Maillard reaction is an important one for the food industry because it produces the melanoidin compounds that give fried, roasted and baked products their colour, and a host of volatiles that impart aroma and flavour. It is multi-step, with amino groups participating in the first stage and the last, and is not one reaction but many. In the final stages, amino acids are deaminated and decarboxylated to give aldehydes (Strecker degradation) and the major route for acrylamide formation is a Strecker-type reaction involving asparagine (Mottram et al., 2007; Halford et al., 2011). Asparagine concentration is the limiting factor for acrylamide formation in heated flour from wheat and rye grain (Muttucumaru et al., 2006; Granvogl et al., 2007; Curtis et al., 2009; 2010). Asparagine accumulates to high concentrations in plants in response to a variety of environmental and biotic stimuli (Curtis et al., 2009; 2010; Lea et al., 2007); in wheat, sulphur deprivation has a particularly dramatic effect, causing increases of up to 30-fold in free asparagine concentration in the grain (Muttucumaru et al., 2006; Granvogl et al., 2007; Curtis et al., 2009). A two-thirds reduction in free asparagine concentration in wheat grain and a mitigation of the effects of sulphur deficiency would be of great benefit to the food industry.

The expression of a similar suite of genes in a gcn2-mutant of Arabidopsis showed little change with wild-type (Zhang et al., 2008). However, the Arabidopsis study did not include an over-expression experiment, or use sulphur deprivation to perturb the system. Nor did it include an analysis of AS1 or SDI1 genes and it was these that differed most between the TaGCN2 over-expressing lines and the controls. Wheat appears to be extremely sensitive to sulphur deprivation and to respond with dramatic changes in free amino acid, particularly free asparagine, accumulation in the grain. The wheat system may, therefore, simply be a better one for demonstrating the role of TaGCN2 in regulating gene expression.

The transgenic plants may have been compensating for TaGCN2 over-expression by increasing expression of eIF2α, the substrate for GCN2-type protein kinases, and of a protein phosphatase 2A, which reverses the action of GCN2. This may explain why there was no evidence of a negative effect on yield in the over-expressing lines. The fact that there were such profound effects on expression of other genes despite this leads us to speculate that GCN2 regulates gene expression in plants through a different mechanism from that described in budding yeast. In that organism, eIF2α phosphorylation by GCN2 controls the translation of transcription factor, GCN4. However, no GCN4 homologue has been identified in plants, despite the extensive genome data that is now available (Halford, 2006). Animals, on the other hand, do have a GCN4 homologue, ATF4, but lack the ability to make many amino acids.

There are other differences between the regulatory system in yeast and the one that is being elucidated in plants. For example, over-expression of TaGCN2 repressed expression of genes encoding AS1, DHS and CGS, increased that of genes encoding AK/HSDH, PSP and DHDPS and had no consistent significant effect on genes encoding AAT, AlaAT, ALS, HDH and PAT. It is evident that this is not the same as the general amino acid control system of yeast, in which activation of GCN2 results in translation of GCN4 and the promotion of expression of genes encoding enzymes in every amino acid biosynthetic pathway except cysteine (Natarajan et al., 2001). The involvement of GCN2 in regulating genes in response to sulphur availability has also never been demonstrated in fungi. Clearly, while some of the components and mechanisms of the regulatory systems controlling protein synthesis and the expression of genes encoding enzymes of amino acid biosynthesis have been conserved as fungi and plants have diverged, others have changed substantially.

In light of the foregoing general description and the specific examples which follow, it will be appreciated that we have shown significant effects as a result of manipulating the regulatory protein kinase, GCN2, on yield, free amino acid concentration and protein content of wheat. We anticipate the same results in other plants, including, but not limited to, oilseed rape, maize, potatoes, and other plants.

While the data provided herein focuses on transgenic wheat lines in which expression of a protein kinase, GCN2, was inhibited by RNA interference (RNAi), and lines in which GCN2 was over-expressed, those skilled in the art will appreciate that adopting similar strategies in other plants will yield similar results. Over-expressing lines evidence a trend toward a higher grain number and yield than controls, and low concentrations of free amino acids in the grain; conversely the RNAi lines have increased concentrations of free amino acids. Increased yield and low free amino acid concentration are desirable traits. The GCN2-overexpressing wheat lines were grown in a containment glasshouse in a randomised design. It was discovered that the RNAi lines were not homozygous, possibly indicating that homozygosity is not achievable with this event. However, three homozygous over-expressing lines were identified.

There were significant increases in total free amino acid concentrations in the seeds of the RNAi plants with free asparagine and glutamine concentration in particular being much higher than in controls, while free amino acid concentrations in the grain of the over-expressing lines showed the opposite trend. Over-expression of TaGCN2 resulted in increases in expression of eIF2α and protein phosphatase PP2A, possibly to compensate for the increase in TaGCN2 activity. However, the expression of several key genes in nitrogen assimilation and amino acid biosynthesis was also affected. There was a significant increase in expression of a nitrate reductase gene and of genes encoding phosphoserine phosphatase and dihydropicolinate synthase, while expression of an asparagine synthetase (AS) gene and genes encoding cystathionine gamma-synthase and sulphur-deficiency-induced-1 (SDI1) all decreased significantly. In sulphur-deprived wild-type plants, SDI1 and AS gene expression increased significantly, but this sulphur deficiency-induced activation of these genes did not occur in the TaGCN2 over-expressing lines at all. The expression of two other genes, encoding aspartate kinase/homoserine dehydrogenase and 3-deoxy-D-arabino-heptulosonate-7-phosphate synthase, was also significantly lower than in controls under sulphur deprivation.

The yield of the over-expressing lines continued to be higher than that of non-transgenic controls (average 26 g grain per plant compared with 19 g for controls in one experiment) but statistical significance was not established. We anticipate similar yield improvement in the harvested organs of other crop plants in which the technology is applied.

The examples below demonstrate that GCN2 plays an important role in the regulation of genes encoding enzymes of amino acid biosynthesis in wheat and implicated GCN2 in sulphur signalling. We believe this is the first demonstration of a clear role for a GCN2-type protein kinase in the regulation of genes encoding enzymes of amino acid biosynthesis in plants, and the first to implicate GCN2-type protein kinases so deeply in sulphur signalling in any organism.

As shown in the examples, transgenic wheat plants were produced which over-expressed TaGCN2 or which contained an RNA interference (RNAi) construct to reduce TaGCN2 gene expression. TaGCN2 gene expression in the RNAi lines was analysed by real-time quantitative PCR using total RNA from developing endosperm as a template and cyclophilin as a reference gene, and five RNAi lines, 122, 138, 140, 208 and 215, showing a reduction in TaGCN2 gene expression, were identified. The reduced expression of an already not very highly expressed gene made this analysis difficult and statistical significance was not established. The data were therefore used for guidance only. Nevertheless, the apparent reduction in expression of TaGCN2 in these lines was consistent with the results of analyses of free amino acids, the levels of which were significantly (p<0.05) higher than in controls. In the case of line 215 the increase was more than 12-fold and this line in particular had accumulated a huge quantity of free asparagine (36.5 mmol kg⁻¹ compared with 3.9 mmol kg⁻¹ in the control) and, even more dramatically, free glutamine (130 mmol kg⁻¹ compared with 0.235 mmol kg⁻¹ in the control). Analysis of the grain from the T3 generation showed that not all the grain contained the transgene. Indeed, a segregation ratio of approximately 1:1 was still apparent, suggesting that it might not be possible to produce homozygous lines from these transgenic events.

Homozygous lines from three independent TaGCN2 over-expressing lines, 395, 402 and 426, were identified. Real-time PCR analysis showed all three to have higher levels of TaGCN2 expression than controls. Free amino acid concentrations in the seeds of these plants showed the opposite trend to those in the RNAi lines. Total free amino acids were significantly reduced (p<0.05) and all three lines showed significant reductions (p<0.05) in asparagine concentration. In over-expressing line 426, free asparagine concentration was 0.955 mmol kg⁻¹, compared with an average of 3.30 mmol kg⁻¹ in the controls, representing a reduction of more than 70%.

Effects of manipulating TaGCN2 gene expression on genes of amino acid biosynthesis under adequate nutrient supply and in response to sulphur deprivation are also disclosed herein. The transgenic wheat lines over-expressing TaGCN2 were used to investigate the role of TaGCN2 in regulating expression of key genes in amino acid metabolism under conditions of sulphur sufficiency and deficiency. The over-expressing lines were homozygous and had reduced levels of free asparagine and other amino acids compared with controls.

Transgenic and control wheat plants were grown in vermiculite, which does not retain nutrients, and feeding was started three weeks after potting. There were two feeding regimes: one set of plants (S+) were watered with ‘complete’ medium containing sufficient amounts of potassium, phosphate, calcium, magnesium, sodium, iron, nitrate and sulphate ions (1.1 mM MgSO₄) (Muttucumaru et al., 2006; Curtis et al., 2009); a second set (S−) was watered with the same medium containing one tenth the concentration of MgSO₄. Sulphur feeding was used to perturb the system in this way because sulphur deprivation causes a dramatic increase in free amino acid levels in wheat grain (Muttucumaru et al., 2006; Granvogl et al., 2007; Curtis et al., 2009).

The expression levels of TaGCN2 and a suite of other genes in flag leaves of two of the over-expressing lines, 402 and 426, were compared with those in wheat cv. cadenza controls by real time, quantitative PCR using genes encoding glyceraldehyde 3-phosphate dehydrogenase (GAPDH) and succinate dehydrogenase (SDH) as reference genes. The target gene sequences were identified initially through BLAST searches of wheat ESTs using annotated Arabidopsis gene sequences. The wheat ESTs were aligned into contigs and checked against rice and Brachypodium genome data. In some cases, the derived cDNA nucleotide sequence was split into its exons and each exon was used in a BLAST search of the wheat genome database (www.cerealsdb.uk.net/search_reads.htm). Contigs were then assembled and manually extended through a sequence of BLAST searches until the full-length gene sequence was obtained.

Statistical analysis of the data assessed the significance of line and sulphur treatment as main effects and the interaction between these two factors using the F-test. TaGCN2 was confirmed to be significantly (p<0.05) over-expressed in both transgenic lines. Expression of eIF2α and protein phosphatase-2A (PP2A) was also significantly increased, possibly indicating that the plants were compensating for the over-expression of TaGCN2 by producing more substrate and more of the opposing phosphatase. There was also a significant (p<0.05) increase in expression of the nitrate reductase (NR) gene. This is consistent with the finding of Zhang et al. (2008) that expression of a nitrate reductase gene was reduced in an Arabidopsis mutant lacking GCN2, and is therefore further evidence of a role for GCN2 in regulating nitrogen assimilation in plants.

In the plants that were supplied with sulphur there were significantly (p<0.05) higher levels of expression of genes encoding phosphoserine phosphatase (PSP) and dihydropicolinate synthase (DHDPS) in the transgenic lines compared with controls, while expression of an asparagine synthetase (AS) gene and genes encoding cystathionine gamma-synthase (CGS) and sulphur-deficiency-induced-1 (SDI1) were all significantly (p<0.05) lower. SDI1 is involved in the utilisation of stored sulphate pools under S-limiting conditions and is used as a marker for sulphur deficiency, while CGS is involved in the synthesis of the sulphur-containing amino acids, cysteine and methionine, as well as other aspects of sulphur metabolism. AS has not previously been shown directly to be sulphur-responsive but asparagine does accumulate to very high concentrations in the grain of sulphur-deprived wheat (Muttucumaru et al., 2006; Granvogl et al., 2007; Curtis et al., 2009).

This link with sulphur was dramatically confirmed by the analysis of the sulphur-deprived plants. In the control lines, the expression of genes encoding SDI1 and AS increased significantly (p<0.05), whereas in the GCN2 over-expressing lines there was no increase in expression at all. The expression of two other genes, encoding aspartate kinase/homoserine dehydrogenase (AK/HSDH) and 3-deoxy-D-arabino-heptulosonate-7-phosphate synthase (DHS), was significantly (p<0.05) lower in the transgenic lines than in controls under sulphur deprivation. AK/HSDH is a bifunctional enzyme but the phosphorylation of aspartate by its aspartate kinase (AK) activity is the first step in methionine, lysine and threonine synthesis. DHS is involved in the early stages of aromatic amino acid synthesis. Expression of PSP in the control plants increased significantly (p<0.05) in response to sulphur deprivation to the levels seen in the over-expressing plants, which did not change in response to sulphur. In other words, over-expression of TaGCN2 resulted in expression of PSP being at the levels seen in sulphur-deprived control plants whether sulphur was supplied or not.

A trend for the over-expressing lines to yield more grain weight per plant than the control plants has already been noted herein. Individual grain weight was similar in control and transgenic lines, indicating that there was no difference in the size of individual grains. The nitrogen content of the grain from the transgenic lines was lower than that of controls, while the carbon content was unchanged, meaning that the transgenic lines had a higher ratio of carbon to nitrogen than the controls. This trend was maintained in the experiments performed in this study, but the yield was relatively low across the board. Overall, statistical significance has still not been established and it may require a field trial to confirm this result.

Nucleotide sequence data for wheat from the UK's wheat genome project became available recently, enabling the identification of wheat DNA encoding eIF2α. A BLAST search of the database (www.cerealsdb.uk.net/search_reads.htm) was performed using a maize eIF2α nucleotide sequence and overlapping contigs were assembled. The derived amino acid sequence of the protein comprised 340 residues with 95-97% amino acid sequence identity with maize, sorghum and rice eIF2α proteins. The putative target residue for phosphorylation by GCN2-type protein kinases was readily identifiable at position 50 of the wheat eIF2α protein.

The data provided herein clearly shows TaGCN2 to be involved in the regulation of gene expression under normal conditions, and also implicates TaGCN2 in sulphur signalling. The involvement of GCN2 or a related protein kinase in sulphur signalling has not been demonstrated clearly in any organism before. The effect of TaGCN2 over-expression on yield and free amino acid content, with its implications for product quality and safety, are of commercial significance. The data provided herein also provides a contribution to knowledge on GCN2-type protein kinases, the impact of which will go beyond crop science to the study of other organisms.

Accordingly, this invention provides a method for modulating the amount of free amino acids, in particular free asparagine, produced in a plant which comprises over-expressing GCN2 in the plant. Thus, in a first aspect, the invention relates to a method for reducing the amount of one or more free amino acid in a plant which comprises introducing and over-expressing a nucleic acid construct encoding GCN2 or a functional variant thereof in said plant.

In one embodiment, the total amount of free amino acids is reduced. In another embodiment, the total amount of free asparagine is reduced. The reduction is compared to a non-transformed control wild type plant which does not overexpress a GCN2 nucleic acid sequence. Reduction of the total amount of free amino acids or the total amount of free asparagine is by at least 10%, for example by about 10% to about 80%, for example about 10%, about 20%, about 30%, about 40%, about 50%, about 60%, about 70% or about 80%.

According to the different aspects and embodiments of the invention described herein, the plant into which a GCN2 nucleic acid sequence of plant or other origin is introduced may be any monocot or dicot plant. Preferably, the plant is a crop plant. By crop plant is meant any plant which is grown on a commercial scale for human or animal consumption or use. For example, the plant may be a cereal crop. In one embodiment, the plant is selected from wheat, rice, barley, maize, oat sorghum, potato, millet, rye, buckwheat, soybean or sugarcane. Preferred plants are maize, wheat, rice, barley or potato.

According to the different aspects of the invention, a nucleic acid construct encoding GCN2 or a functional variant thereof is introduced and overexpressed in a plant. A functional variant of GCN2 is a peptide that retains the biological activity of the non-variant GCN2, and leads to, when overexpressed, a reduction in the total amount of free amino acid as described herein. For example, the functional variant may be a homologue or orthologue of TaGCN2 as shown in SEQ ID NO:1. The functional variant of GCN2 may a peptide that comprises alterations/mutations in its sequence when compared to the wild type sequence, but these mutations do not affect the biological function of the peptide.

In one embodiment, the nucleic acid construct encodes a plant GCN2. The gene encoding GCN2 and which is introduced and overexpressed in a plant may be an exogenous gene, such as AtGCN2 or TaGCN2 overexpressed in a different plant species.

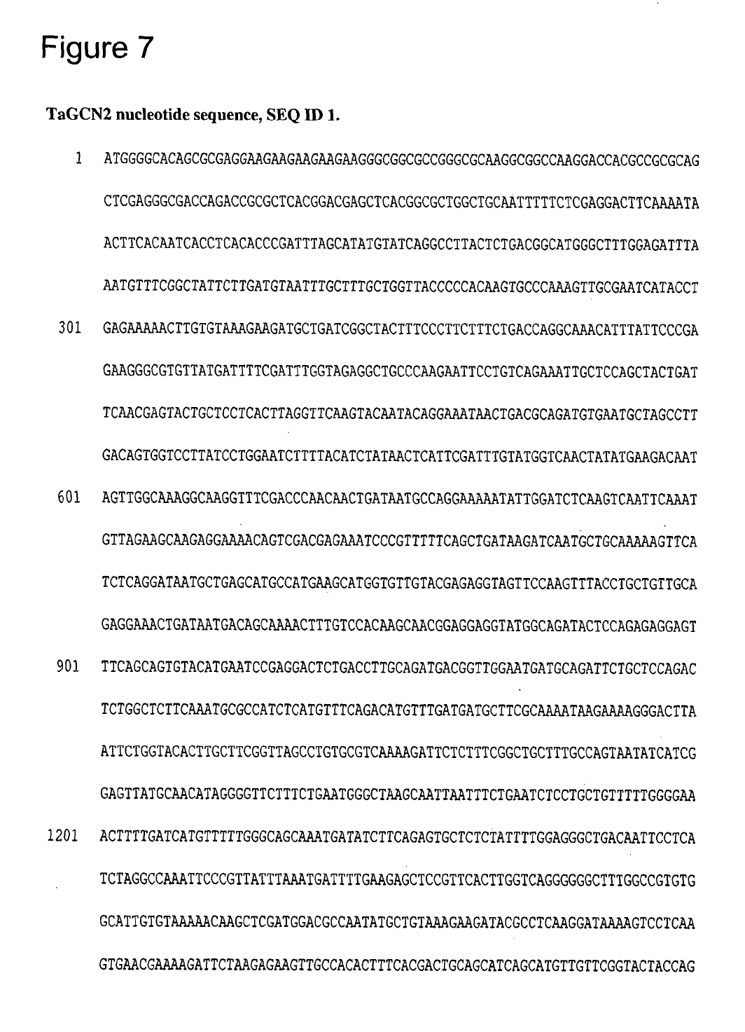

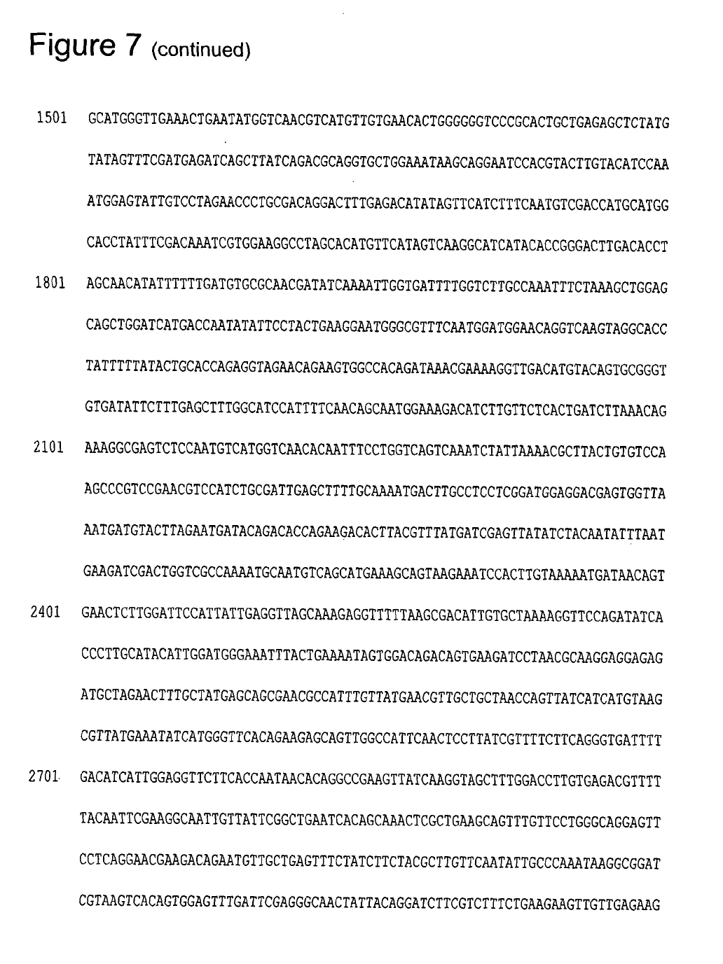

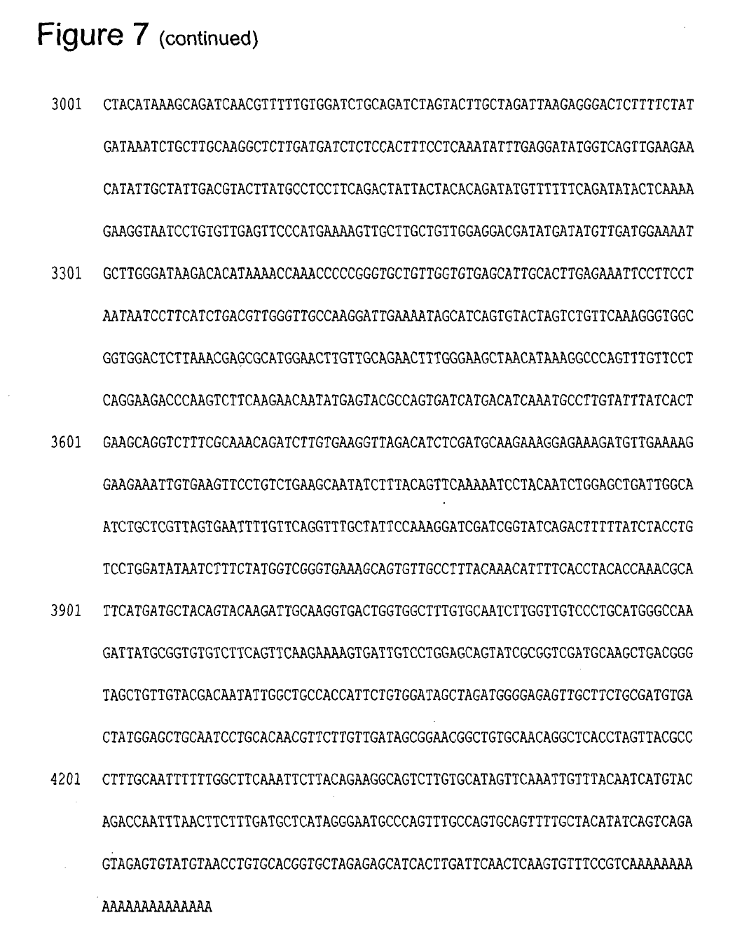

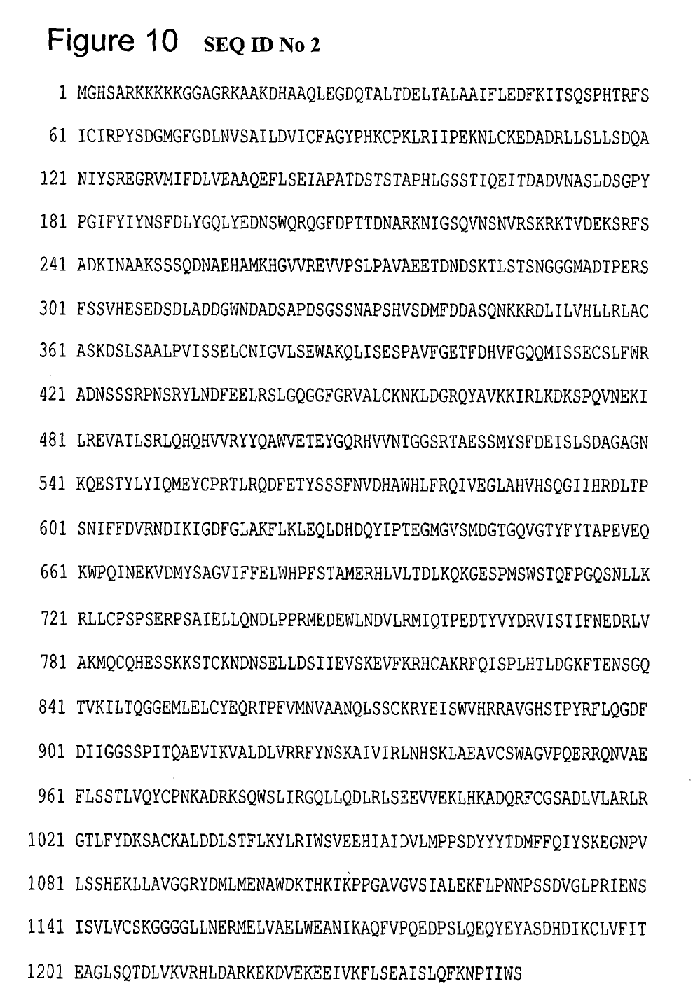

Thus, in one embodiment of the different aspects of the invention, the exogenous plant GCN2 may originate from any plant, for example a family or species listed above and expressed in a different plant species according to the invention. For example, homologues and orthologues of AtGCN2 or TaGCN2 as shown in SEQ ID NO:1 (FIG. 7) or its polypeptide sequence SEQ ID NO:2 (FIG. 10) can be derived from any plant as long as the homologue confers the herein mentioned activity, i.e. it is a functional equivalent of said molecule. The homologue of the TaGCN2 gene or polypeptide shown in SEQ ID NO:1 and SEQ ID NO:2, respectively, has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid or amino acid represented by SEQ ID NO:1 or 2. The overall sequence identity is determined using any alignment algorithms known in the art. Homologues or orthologues can thus be identified in other species using bioinformatics. Examples of homologous sequences are shown in example 7. The polypeptide homologues or orthologues preferably comprise motifs characteristic for GCN2.

Alternatively, the plant GCN2 may be an endogenous plant GCN2, i.e. a plant GCN2 that is endogenous to the plant in which it is introduced and overexpressed. For example, in one embodiment, TaGCN2 is overexpressed in wheat. In this embodiment, a construct comprising SEQ ID NO:1 is expressed.

However, the various aspects of the invention are not limited to the use of any plant GCN2 and also extend to the use of any fungal or animal GCN2. GCN2 from different organisms are highly conserved and GCN2 from one organism can therefore function in a different organism. For example, plant eIF2alpha is phosphorylated by yeast GCN2, at least when expressed in yeast, and the target site in eIF2alpha appears to be tightly conserved throughout the eukaryotes.

The reduction of the total amount of one or more free amino acids may be observed in any plant part. In one embodiment, the reduced amount of one or more free amino acids is in the seed/grain of a plant.

All nucleic acid constructs as described herein may further comprise a regulatory sequence. Thus, the nucleic acid sequence(s) described herein may be under operative control of a regulatory sequence which can control gene expression in plants. A regulatory sequence can be a promoter sequence which drives the expression of the gene or genes in the construct. Preferably, the nucleic acid sequence is expressed using a promoter that drives overexpression. Overexpression according to the invention means that the transgene is expressed at a level that is higher than expression of endogenous counterpart (endogenous plant GCN2) driven by the endogenous promoter. For example, overexpression may be carried out using a strong promoter, such as the cauliflower mosaic virus promoter (CaMV35S), the rice actin promoter or the maize ubiquitin promoter or any promoter that gives enhanced expression. Alternatively, enhanced or increased expression can be achieved by using transcription or translation enhancers or activators and may incorporate enhancers into the gene to further increase expression. Furthermore, an inducible expression system may be used. Also, expression systems that direct expression of the constructs described herein in specific plant parts, for example seeds, may be used. Other suitable promoters and inducible systems are also known to the skilled person.

As a skilled person will know, the construct may also comprise a selectable marker which facilitates the selection of transformants, such as a marker that confers resistance to antibiotics, such as kanamycin.

The constructs described herein may be part of a vector.

The invention also provides such a transgenic plant in which GCN2 is overexpressed. Thus, the invention relates to a transgenic plant with a reduced amount of one or more free amino acids wherein said plant overexpresses a gene encoding GCN2 or a functional variant thereof.

In one embodiment, the plant is obtained or obtainable by the methods described herein.

The invention also extends to harvestable parts of a transgenic plant according to the invention such as, but not limited to seeds, leaves, fruits, flowers, stems, roots, rhizomes, tubers, and bulbs, which harvestable parts comprise a recombinant nucleic acid encoding a GCN2 polypeptide. The invention furthermore relates to products, such as food products derived, preferably directly derived, from a harvestable part of such a plant.

For the purposes of the invention, the use of the terms “transgenic”, “transgene” or “recombinant” means (with regard to, for example, a nucleic acid sequence, an expression cassette, gene construct, vector comprising the nucleic acid sequence or a plant transformed with the GCN2 nucleic acid sequences, expression cassettes or vectors according to the invention) that methods are used in which the nucleic acid sequence encoding the GCN2 proteins useful in the methods of the invention are not located in their natural genetic environment or have been modified by recombinant methods. The natural genetic environment is understood as meaning the natural genomic or chromosomal locus in the original plant.

A transgenic plant for the purposes of the invention is thus understood as meaning, as above, that the nucleic acids used in the method of the invention are not at their natural locus in the genome of said plant. Preferred families and species of plants are mentioned herein.

In certain embodiments, the nucleic acid sequence encoding the GCN2 proteins useful in the methods of the invention have been modified by recombinant methods to lead to a dominant mutant which, when expressed, results in a reduced level of free amino acids in a plant.

Methods for plant transformation to introduce the GCN2 nucleic acid sequences as described herein, for example by Agrobacterium mediated transformation or particle bombardment, and subsequent techniques for regeneration and selection of transformed plants are well known in the field. Also within the scope of the invention is chloroplast transformation through biobalistics.

In another aspect, the invention also provides a method for reducing the amount of acylamide produced upon processing of plant material which comprises introducing into and over-expressing in a plant a nucleic acid construct operatively encoding GCN2, from which plant material is to be obtained for processing. In such method, the plant produces reduced amounts of asparagine, preferably but not exclusively, in the seed, tuber or other harvestable organ of the plant. The invention thus provides a method for using GCN2 to produce a plant with reduced asparagine, or to limit plant responses to low sulphur or sulphur starvation which might negatively impact on food safety, which comprises introducing into and over-expressing in the plant a nucleic acid construct operatively encoding GCN2. In an alternate formulation of the invention, it provides a method for producing grain of improved quality by reducing the formation of carcinogenic chemicals during heat processing which comprises introducing into and over-expressing in a plant a nucleic acid construct operatively encoding GCN2.

In another aspect, the invention relates to a method for producing a plant with a reduced amount of one or more free amino acids, which comprises introducing into and over-expressing in a plant a nucleic acid construct encoding GCN2.

The invention also relates to the use of a nucleic acid comprising a GCN2 nucleic acid sequence or a functional variant thereof to reduce the level of one or more free amino acids in a plant. As described above, the GCN2 sequence may be of plant, fungal or animal origin. In one embodiment, the GCN2 sequence is of plant origin. For example, the GCN2 sequence comprises or consists of SEQ ID NO:1. In another embodiment, the sequence is a functional variant, homologue or orthologue of SEQ ID NO:1. As explained above, the homologue of the TaGCN2 gene or polypeptide shown in SEQ ID NO:1 or 2 has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid or amino acid represented by SEQ ID NO:1 or 2.

In one embodiment, said sequence is used to reduce free asparagine in a plant, for example in the seed or tuber.

In yet another aspect, the invention relates to a method for producing plants with increased yield which comprises introducing into and overexpressing in a plant a nucleic acid construct encoding GCN2.

In a further aspect, the invention relates to a method for producing plants with increased abiotic stress tolerance which comprises introducing into and over-expressing in a plant a nucleic acid construct encoding GCN2.

In a further aspect, the invention relates to a method for modulating a plant response to sulphur deprivation or starvation which comprises introducing into and over-expressing in a plant a nucleic acid construct encoding GCN2.

The invention also includes methods for the production of a product comprising a) growing the plants of the invention and b) producing said product from or by the plants of the invention or parts, including seeds, of these plants. In a further embodiment the methods comprises steps a) growing the plants of the invention, b) removing the harvestable parts as defined above from the plants and c) producing said product from or by the harvestable parts of the invention. In one embodiment the products produced by said methods of the invention are plant products such as, but not limited to, a foodstuff, such as flower, feedstuff, food supplement or feed supplement.

### EXAMPLES

While the foregoing disclosure generally describes the subject invention, including how to make and use the invention such that those skilled in the art are enabled to practice this invention, including its best mode, the following examples are provided by way of ensuring the complete and enabling written description of this invention. The specifics of these examples should not, however, be taken as limiting on the invention. Rather, for that purpose, reference should be had to the appended claims and the equivalents thereof.

In the Examples that follow, unless specified to the contrary, the following Materials and Methods were employed:

Isolation of Wheat Leaf RNA

Six-week-old wheat (Triticum aestivum cv. Cadenza) leaf material was snap frozen in liquid nitrogen before being crushed to a fine powder using a chilled pestle and mortar. Total RNA was purified using the RNeasy Mini Kit (Qiagen Ltd, Crawley, UK) following the manufacturer's instructions. Alternatively, RNA was extracted from leaf material with Trizol reagent (Invitrogen Ltd, Paisley, UK). RNA was treated with DNase (Promega, Southampton, UK) to prevent DNA contamination. RNA quality was checked using a spectrophotometer and in some cases by agarose gel electrophoresis.

Isolation of RNA from Grain

Up to 250 mg of frozen grain material was allowed to thaw momentarily, squashed to rupture the structure, then re-frozen in liquid nitrogen and ground to a fine powder. RNA was extracted from powdered grain tissue using the CTAB method (Chang et al., 1993). RNA was further purified using the RNeasy MinElute clean up column that included an on-column DNase treatment (Qiagen, Crawley, UK).

Molecular Cloning of TaGCN2

The design of the antisense primer used to amplify a product from TaGCN2 mRNA (GCCAATCAGCTCCAGATTGTAGGA (SEQ ID NO:3)) was based on a wheat expressed sequence tag (EST) matching the 3′ end of the Arabidopsis AtGCN2 nucleotide sequence (Zhang et al., 2003), which was identified using an in-silico search of the WhETS database (Mitchell et al., 2007). A similar search revealed a previously uncharacterised rice (Oryza sativa) GCN2-like nucleotide sequence (GENBANK: XM473001) which was used to design a sense primer: ATGGGGCACAGCGCGAGGAAGAAGAA (SEQ ID NO:4).

Complementary-DNA was generated from the wheat RNA by reverse transcription using SuperScript III (Invitrogen, Paisley, UK). Amplification by PCR used Phusion High-Fidelity DNA Polymerase (Finnzymes, Vantaa, Finland). Cycling conditions were: 98° C. for 30 s; 40 cycles of 98° C. for 10 s, 50° C. to 70° C. gradient for 20 s, and 72° C. for 3 min.; final hold at 72° C. for 10 min.

PCR products were cloned and nucleotide sequences were determined using a BigDye Terminator v3.1 Cycle Sequencing kit (Applied Biosystems, Life Technologies, Carlsbad, Calif., USA). The reaction conditions were: 96° C. for 1 min., followed by 25 cycles of 96° C. for 10 s, 50° C. for 5 s and 60° C. for 4 min. Nucleotide sequence analysis was performed by Geneservice, Source Bioscience (Nottingham, UK) or MWG Biotech (Wolverhampton, UK).

Amplification of 3′ cDNA Ends

The nucleotide sequence of the 3′ end of the TaGCN2 transcript was determined by rapid amplification of the cDNA end (RACE) using a GeneRacer kit (Invitrogen), which incorporates Phusion High-fidelity DNA polymerase. Three primers were used: AGTCTGTTCAAAGGGTGGCGGTGG (SEQ ID NO:5), GGTGGACTCTTAAACGAGCGCATGGA (SEQ ID NO:6), and ACCAATAACACAGGCCGAAG (SEQ ID NO:7). The PCR conditions were as follows: 98° C. for 30 s; 40 cycles of 98° C. for 10 s, 65° C. for 15 s and 72° C. for 20 s; final extension at 72° C. for 10 min. An aliquot of the reaction product was analysed by agarose gel electrophoresis; another aliquot was then used as the template for nested PCR.

Production of Transgenic Wheat Plants.

In order to produce TaGCN2 over-expressing plants, the full-length TaGCN2 open reading frame was spliced into a plasmid downstream of a rice actin gene promoter, which is constitutively active (McElroy et al., 1990). The termination signal was from the Agrobacterium tumefaciens nopaline synthase gene (nos) (Jefferson, 1987). The plasmid was introduced into wheat by particle bombardment of scutella tissue. Plasmid pAHC20 (Christensen and Quail, 1996), which conveys resistance to the herbicide phosphinothricin (PPT), was used for co-transformation. Following selection, PCR was used to establish the presence of the transgene. Transgenic plants were self-fertilised and PPT-resistant progeny that tested positive for the presence of the construct were selected. This was repeated to the T3 generation and three independent, homozygous lines, 395, 402 and 426, were produced. Embryo isolation and bombardment and plant regeneration and selection were performed within the Rothamsted Cereal Transformation Laboratory using the methods described by Sparks and Jones (Sparks and Jones, 2009). The presence of the transgene in individual plants was checked by PCR using genomic DNA as the template. The primers used were 5′-CAAGGACCACGCCGCGCAG (SEQ ID NO:8), which anneals in exon 1, and 5′-GCTAAATCGGGTGTGAGGTGATTGTG (SEQ ID NO:9), which anneals in exon 2. The product amplified from the endogenous gene therefore contained an approximately 0.8 kb intron that was not present in the transgene. Successive self-fertilisation to the T₃ generation was carried out to achieve homozygosity. For RNA interference (RNAi), a 422 by inverted repeat section of the Ta GCN2 PCR product was inserted into plasmid pHANNIBAL (Wesley et al., 2001). The promoter (from −378 to +24 by with respect to the transcription start site) of a high molecular weight glutenin subunit (HMW subunit) gene (Glu-1D-1) was spliced upstream of the Ta GCN2 DNA in place of the CaMV35S promoter in pHANNIBAL. The HMW subunit gene promoter is endosperm-specific (Lamacchia et al., 2001). The promoter fragment was amplified by PCR using primers ATTTGGCCAGTCGGCCGCGGCCGCGAAGCTTTGAGTGGCCGTAGA (SEQ ID NO:10) and CCGCTCGAGCGGGTGCTCGGTGTTGTG (SEQ ID NO:11), which incorporated restriction sites for SfiI and XhoI at the 5′ and 3′ end of the product, respectively. This enabled the CaMV35S promoter from pHANNIBAL to be excised using these two restriction enzymes and replaced with the HMW subunit gene promoter. The presence of the transgene in individual plants was checked by PCR using genomic DNA as the template. The primers used were 5′-CCAAATAAGGCGGATCGTAAGTCACAG (SEQ ID NO:12) and 5′-CCATGGTCCTGAACCTTCACCTCG (SEQ ID NO:13), which did amplify a product from wild-type DNA.

Sulphur Feeding

Transgenic and control wheat plants were grown in vermiculite in a glasshouse with a 16 hour day-length (supplemental lighting was used as necessary) and a minimum temperature of ° C. Vermiculite does not retain nutrients, so once seed reserves were exhausted the only nutrition available to the developing seedlings came from externally applied liquid feed solution. Feeding was started three weeks after potting and continued every two days until harvest. Distilled water was also supplied as required to prevent water stress. A completely randomised design was used for the pots in the glasshouse. Plants were supplied with either a medium containing a full nutrient complement of potassium, phosphate, calcium, magnesium, sodium, iron, nitrate (2 mM Ca(NO₃)₂ and 1.6 mM Mg(NO₃)₂) and sulfate ions (1.1 mM MgSO₄) (Muttucumaru et al., 2006; Curtis et al., 2009), or the same medium containing one tenth the concentration of MgSO₄. RNA was prepared from flag leaves as described above.

Expression Analyses by Real-Time Quantitative PCR

First strand cDNA synthesis was performed using SuperscriptIII (Invitrogen) to reverse transcribe 1-2 μg DNase-treated RNA and was primed with an anchored dT₂₀ primer in a final volume of 20 μL. The qPCR reaction mix consisted of 10 μL SYBR Green JumpStart Taq ReadyMix (Sigma, Poole, UK), 5 μL diluted cDNA and 5 μL primers (125 nM final concentration). Samples were run in an ABI7500 real-time PCR system (Applied Biosystems) and the amplification conditions were 95° C. for 2 min, then 45 cycles of 95° C. for 15 s followed by 67° C. for 45 s. Primer nucleotide sequences were as follows:

Primers used for expression analyses. Gene details are given in the below Table of Primers. F and R refer to ‘forward’ and ‘reverse’ primers. More than one primer pair was used for some genes.

Table of Primers

| Name | Sequence |
|---|---|
| AATx-02F | GCTTGTAATTAGGCCTATGTATTCGAACCCTC |
| | (SEQ ID NO: 14) |
| AATx-02R | AGCCATGGCCTTCAGCTCCAGAGTC |
| | (SEQ ID NO: 15) |
| AATy-01F | TCCTGAACAGTGGGAGAAACTGGCAG |
| | (SEQ ID NO: 16) |
| AATy-01R | CAGCCTGACAGAAGATGCATCTTCATCAAG |
| | (SEQ ID NO: 17) |
| AATz-01F | ATGGCATACCAAGGATTTGCTAGCGGTG |
| | (SEQ ID NO: 18) |
| AATz-01R | GGATACTCAGGCATCCCGCTCTCTG |
| | (SEQ ID NO: 19) |
| AKHSDH-01F | AGAGATCGTCTCTGATTCTTGAGAAGGCG |
| | (SEQ ID NO. 20) |
| AKHSDH-01R | GCCTCGCTGTCATTCACCACCAAG |
| | (SEQ ID NO: 21) |
| AKHSDH-02F | GCTACTAGTGAAGTCAGCATATCATTGACACTAG |
| | (SEQ ID NO: 22) |
| AKHSDH-02R | GGGAAATGATTGATCTGTGCTGTAGGAGATG |
| | (SEQ ID NO: 23) |
| AlaAT-02F | CTGATGCATTCTATGCTCTTCGTCTCCTTGAG |
| | (SEQ ID NO: 24) |
| AlaAT-02R | GAACGTCTCATGGAACACCGTGAAGCG |
| | (SEQ ID NO: 25) |
| ALS-02F | GCATCAGGAGCACGTGCTGCCTATG |
| | (SEQ ID NO: 26) |
| ALS-02R | ATTGCGCATGTCACACTTGTAGGTCTTGTAG |
| | (SEQ ID NO: 27) |
| AS1-03F | GATAAGATGATGTCAAATGCAAAGTTCATCTACC |
| | (SEQ ID NO: 28) |
| AS1-03R | GCCGTGCTGCATGCAACGCTTG |
| | (SEQ ID NO: 29) |
| AS1-04F | GAGCAGTTCAGTGATGGTGTTGGC |
| | (SEQ ID NO: 30) |
| AS1-04R | GCACCGTCAGGATCGCCGAGTT |
| | (SEQ ID NO: 31) |
| CGS-01F | CATGTCCTACTGGGATTCGAAGGAGCAG |
| | (SEQ ID NO: 32) |
| CGS-01R | CAGGTCCTCAAAATCCT GACTCCGATG |
| | (SEQ ID NO: 33) |
| CyclophilinF | CACCGTCCCCTGCAATTG |
| | (SEQ ID NO: 34) |
| CyclophilinR | AGCCCACCTTCTCGATGTTCT |
| | (SEQ ID NO: 35) |
| DHDPS1-01F | ATTGTCGAAGCCATTGGACG |
| | (SEQ ID NO: 36) |
| DHDPS1-01R | TATTCAATACCTGCTGATCAACAC |
| | (SEQ ID NO: 37) |
| DHDPS1-03F | CATACACTCCCCTCCCTCTTGAGAAGAG |
| | (SEQ ID NO: 38) |
| DHDPS1-03R | GCTGATCAACACGAAATCATCGTCATCCAGAAC |
| | (SEQ ID NO: 39) |
| DHS2-02F | GACCAAGAAGGAAGCCACCCAGGAG |
| | (SEQ ID NO: 40) |
| DHS2-02R | GTCGCAGTGGGTGTGGTAGCGG |
| | (SEQ ID NO: 41) |
| eIF2a-01F | GAGGGCATGATCCTCTTCTCCGAG |
| | (SEQ ID NO: 42) |
| eIF2a-01R | CCCGGCGCTTGGAGAGGTCG |
| | (SEQ ID NO: 43) |
| GAPDH-F | ACCCCTTCATCACCACCGACTACATGACC |
| | (SEQ ID NO: 44) |
| GAPDH-R | GGATCTCCTCAGGGTTCCTGCAGCC |
| | (SEQ ID NO: 45) |
| GCN2-02F | GCGGATCGTAAGTCACAGTGGAGTTTG |
| | (SEQ ID NO: 46) |
| GCN2-02R | CCCTCTTAATCTAGCAAGTACTAGATCTGCAG |
| | (SEQ ID NO: 47) |
| GCN2-03F | GGCTGCTTTGCCAGTAATATCATCGGAG |
| | (SEQ ID NO: 48) |
| GCN2-03R | AGAGAGCACTCTGAAGATATCATTTGCTGCC |
| | (SEQ ID NO: 49) |
| HDH-01F | CGCCCTCGCATTGACTTCACCTCC |
| | (SEQ ID NO: 50) |
| HDH-01R | CACAACGGCATTATCGAGAGTGACCTTG |
| | (SEQ ID NO: 51) |
| NR-01F | GGTACTGGTGCTGGTGCTTCTGG |
| | (SEQ ID NO: 52) |
| NR-01R | GAACCAGCAGTTGTTCATCATGCCCATG |
| | (SEQ ID NO: 53) |
| PAT1-01F | GCGTTCAACGCAAAAGTTCTCCAGGATG |
| | (SEQ ID NO: 54) |
| PAT1-01R | CGGAGCGCTGCGTCTCCTGTG |
| | (SEQ ID NO: 55) |
| PP2A-02F | CTTGAAGCGCCTGGCGGAGGAG |
| | (SEQ ID NO: 56) |
| PP2A-02R | GGATGGTCATGCGATACAGATAATGTGGG |
| | (SEQ ID NO: 57) |
| PSP-01F | GTTCCATTTGAAGAGGCTCTTGCTGCCC |
| | (SEQ ID NO: 58) |
| PSP-01R | CCTGGGTGGCCTCTTCTCCAAACAG |
| | (SEQ ID NO: 59) |
| SDH-01F | GAGACGCTCCATTTGCTCTCCGTG |
| | (SEQ ID NO: 60) |
| SDH-01R | ATCTTCTGTCGCAGAGCTCCTAAGGG |
| | (SEQ ID NO: 61) |
| SDI1ex1-01F | CCGTATGCACGGGCCAAGCAC |
| | (SEQ ID NO: 62) |
| SDI1ex2-01R | GCTGCTTCATCACCACCGCCATG |
| | (SEQ ID NO: 63) |
| SDI1ex2-01F | GGAAGCAGTCGCAGGAGTCCCTG |
| | (SEQ ID NO: 64) |
| SDI1ex3-01R | GCTTGAGCAGCTCGATCTCCTCCTTG |
| | (SEQ ID NO: 65) |
| 1433NR-02F | CTCTATGGACATCCGACACCAATGAGGATG |
| | (SEQ ID NO: 66) |
| 1433NR-02R | ACCTCACTGTCCGTCTCCAGATTCTTTTGG |
| | (SEQ ID NO: 67) |

The efficiencies of the reactions were estimated using the LinReg PCR program (Ramakers et al., 2003), and the ct (at threshold fluorescence) and efficiency values were then used to calculate the normalised relative quantity (NRQ) with respect to the reference genes, cyclophilin, succinate dehydrogenase (SDH) and glyceraldehyde 3-phosphate dehydrogenase (GAPDH), for each sample/target gene combination.

$$N R Q = \frac{{E}_{target}^{- ct , target}}{\sqrt{{E}_{S D H}^{- ct , S D H} * {E}_{GAPDH}^{- ct , GAPDH}}}$$

where E_{target}, E_{SDH} and E_{GAPDH} are the estimated reaction efficiencies for particular target, and the two reference, genes, and where ct,target, ct,SDH and ct,GAPDH are the corresponding ct values.

Statistical analysis of the sulphur feeding experiment data was performed using the GenStat® (2010, Thirteenth Edition, VSN International Ltd, Hemel Hempstead, UK) statistical system. and is described in Example 5. There were three biological replicates (leaf tissue samples) for each line by sulphur treatment combination.

Amino Acid Analyses

Free amino acid concentrations in mature grain of compost-grown plants were determined by gas chromatography-mass spectrometry (GC-MS) using methods described previously (Muttucumaru et al., 2006; Curtis et al., 2009). For each amino acid and the total, wheat lines were compared using analysis of variance (ANOVA). Following an F-test result indicating significant (p<0.05) overall differences between lines, specific comparisons of transgenic lines to controls were made using the standard error of the difference (SED) in post-ANOVA t-tests based on the residual degrees of freedom (df). Analysis was performed using GenStat®.

Total Nitrogen and Carbon Analysis

Measurements of total grain nitrogen and carbon were made by the Analytical Unit of the Soil Science Department, Rothamsted Research, using the ‘Dumas’ digestion method and a LECO CNS 2000 Combustion Analyser (LECO Corporation, Saint Joseph, Mich., USA).

### Example 1

### Molecular Cloning of a Wheat (Triticum aestivum) GCN2 Homologue, TaGCN2

A GCN2-related polymerase chain reaction (PCR) product was amplified from wheat cv. Cadenza leaf RNA. A product of approximately 3.8 kb containing an open reading frame running from bases 1 to 3741 was cloned. This open reading frame encoded a protein having 52% amino acid sequence identity with AtGCN2 (Zhang et al., 2003) and 84% identity with a rice (Oryza sativa) GCN2-type protein kinase encoded by mRNA nucleotide sequence XM473001 from GENBANK. The protein was given the name TaGCN2. Note the significantly higher degree of identity with the other cereal GCN2 homologue than with AtGCN2.

Additional nucleotide sequence data from the 3′ end of the transcript was obtained by rapid amplification of the cDNA end (3′RACE). This showed the TaGCN2 transcript to have a 658 nucleotide un-translated region prior to a poly-adenosine tail of 22 nucleotides. The entire sequence of 4439 nucleotides was submitted to the EMBL data base and has been assigned the accession number FR839672.

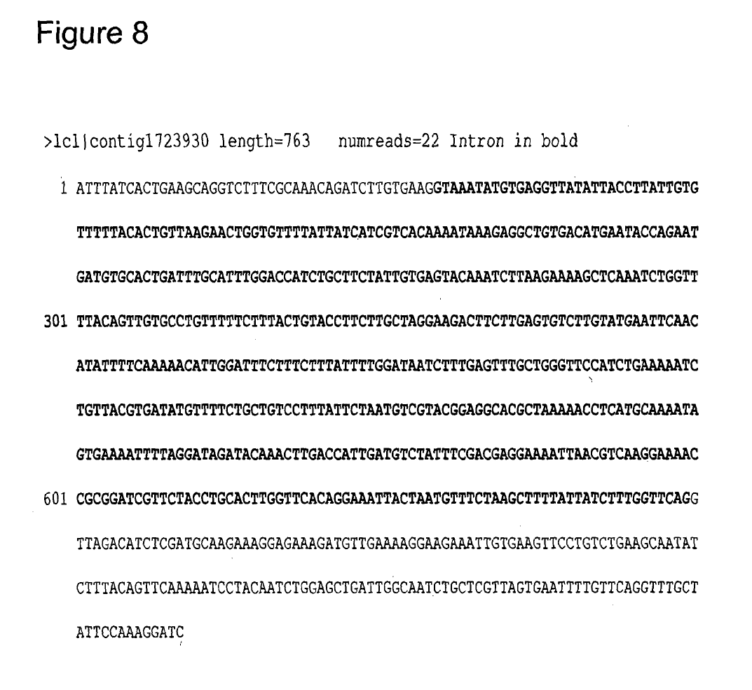

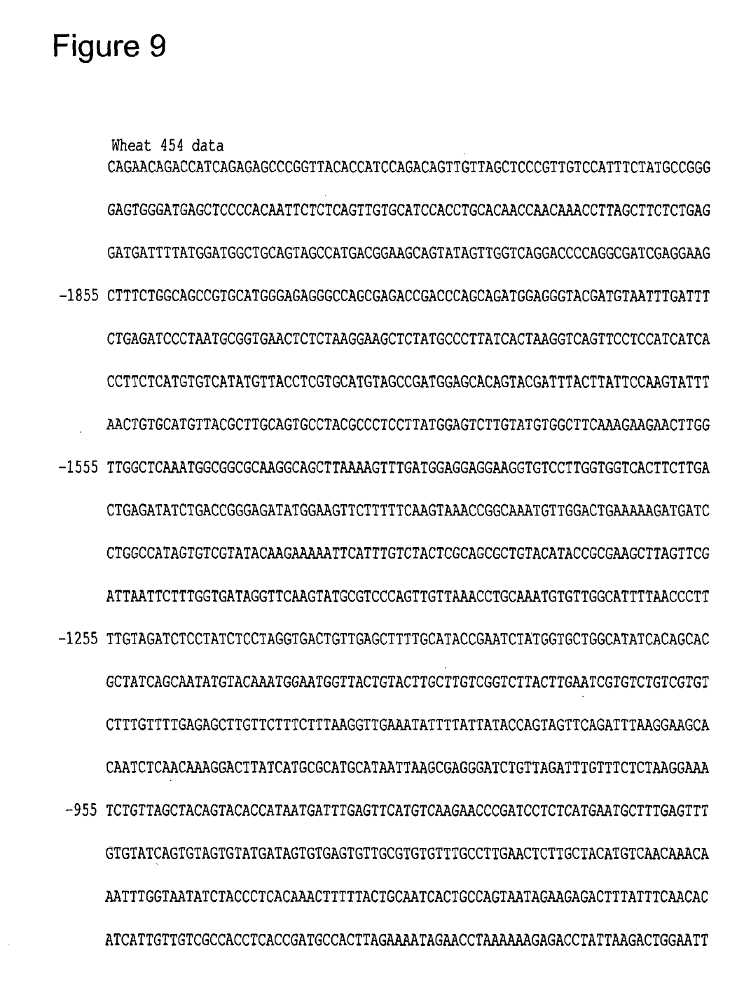

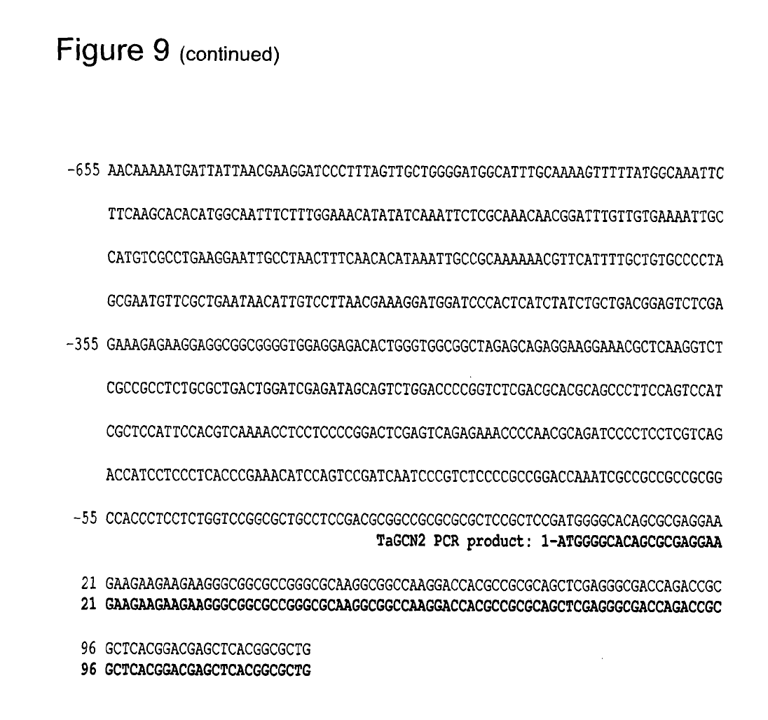

The TaGCN2 nucleotide sequence was used to mine the recently available wheat genomic sequence (www.cerealsdb.uk.net/search_reads.htm) and three separate contigs were identified that matched different parts of the TaGCN2 sequence. The consensus sequence of one of these contigs aligned with the 5′ end of the TaGCN2 PCR product and extended a further 2 kb ‘upstream’ of the ATG translation start site. Another contig aligned with the 3′ end of the TaGCN2 PCR and 3′RACE products, with an intron in the 3′ untranslated region. The entire nucleotide and derived amino acid sequence of the TaGCN2 PCR product and the wheat genome sequence data that aligned with the 5′ and 3′ ends are shown in FIGS. 7-10, which show: the TaGCN2 nucleotide sequence, including the nucleotide sequence of the TaGCN2 PCR product; full nucleotide sequence of wheat genome data (www.cerealsdb.uk.net/search_reads.htm) contig 1723930; alignment of TaGCN2 PCR product with wheat genome data at the 5′ end; derived amino acid sequence for TaGCN2;

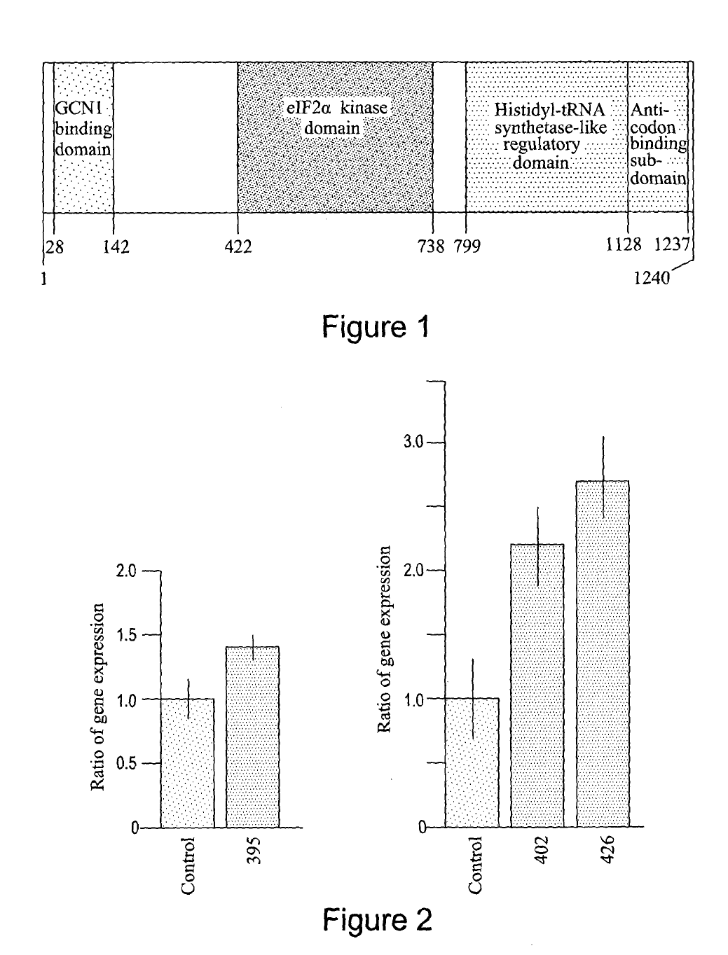

The encoded protein consists of 1247 amino acid residues and has a molecular weight of 140 kDa. It contains a RING-finger, WD40, DEAD-box helicase domain (RWD-domain) at the N-terminus between residues 28 and 142, an eIF2α kinase domain between amino acid residues 422 and 738, and a Histidyl-tRNA synthetase-like regulatory domain towards the C-terminal end of the protein between residues 799 and 1128 (FIG. 1). An anti-codon binding sub-domain of the regulatory domain was found at the extreme C-terminal end of the protein between amino acid residues 1129 and 1237, although it is truncated in TaGCN2 compared with yeast GCN2. The presence of these domains in the same protein is a defining characteristic of GCN2-type protein kinases (Wek et al., 1995).

In Arabidopsis, AtGCN2 has been shown to be expressed in all tissues (Zhang et al., 2003). Expression of TaGCN2 in flag leaves and grain through the period of grain development was analysed by real-time PCR. Transcripts were detectable in all of the samples and no significant changes in transcript levels between tissues or at different developmental stages were evident.

### Example 2

### In Silico Identification of a Wheat (Triticum aestivum) eIF2α Homologue

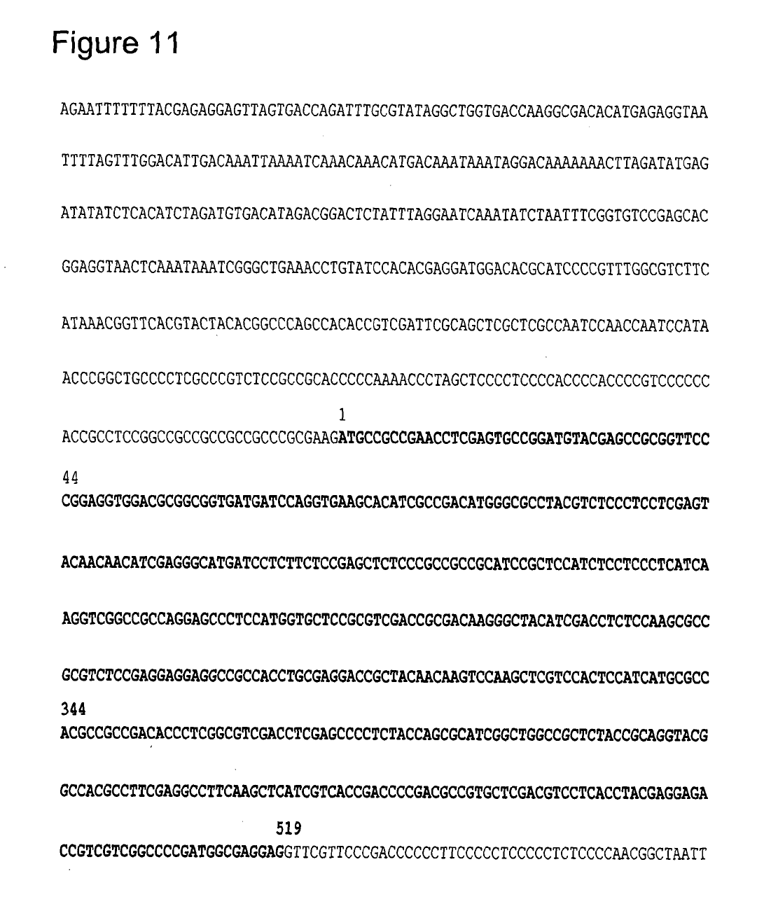

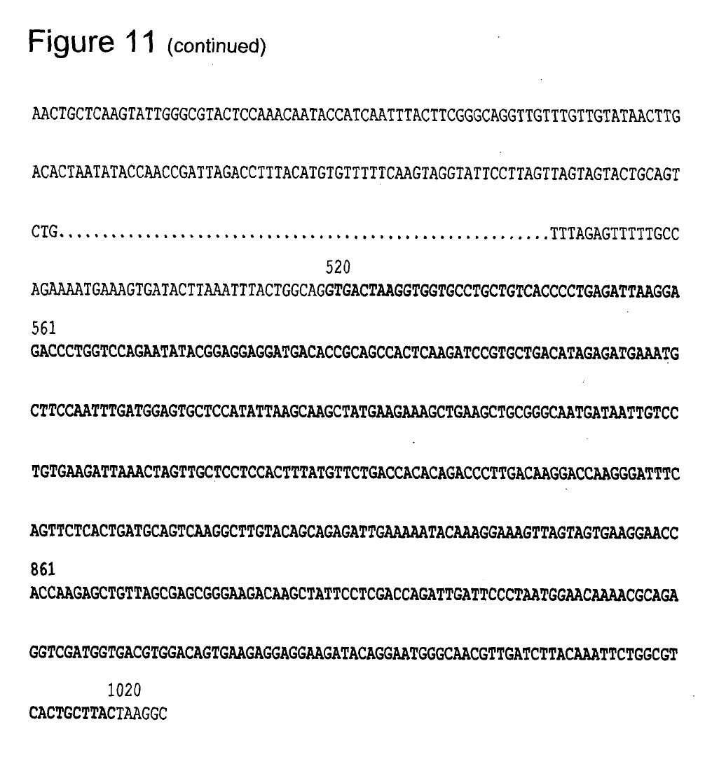

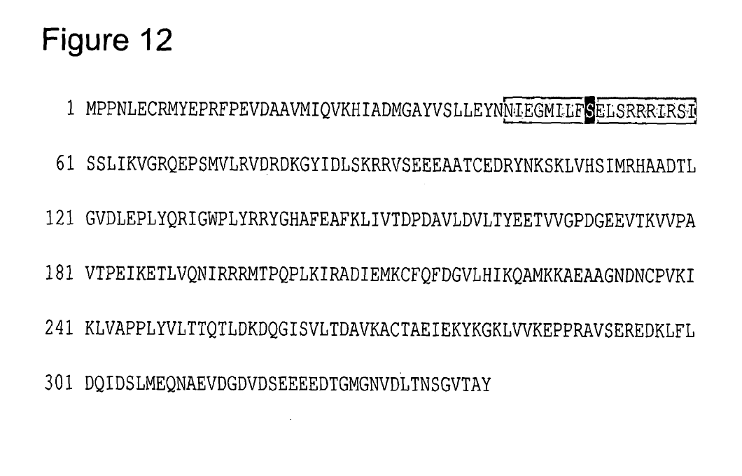

A search of the wheat genome database (www.cerealsdb.uk.net/search_reads.htm) was performed using a maize eIF2α nucleotide sequence, accession NP-001146159, and overlapping contigs were assembled. The nucleotide sequence is shown in FIG. 11 and the derived amino acid sequence of the encoded protein is shown in FIG. 12; the protein comprised 340 amino acids with 95-97% amino acid sequence identity with maize, sorghum and rice eIF2α proteins, accession numbers ACL53376, EER95021 and ABF95443. The putative target residue for phosphorylation by GCN2-type protein kinases is a serine residue in the N-terminal region and it was readily identifiable at position 50 of the wheat eIF2α protein (highlighted in FIG. 12). This and the surrounding residues are absolutely conserved in organisms as diverse as yeast, humans and plants: NIEGMILFSELSRRRIRSI (SEQ ID NO:69) (target serine in bold and underlined).

### Example 3

### Production of Transgenic Wheat Plants Over-Expressing TaGCN2

TaGCN2 was over-expressed in transgenic wheat plants under the control of a rice actin gene promoter, which is constitutively active (McElroy et al., 1990). Three independent, homozygous lines, 395, 402 and 426, were produced. These lines came from separate transformation experiments and TaGCN2 expression in 395 was measured using cyclophilin as a reference gene, while that in 402 and 426 was measured using glyceraldehyde 3-phosphate dehydrogenase (GAPDH) and succinate dehydrogenase (SDH) as reference genes. All three showed higher levels of TaGCN2 expression than controls (FIG. 2).

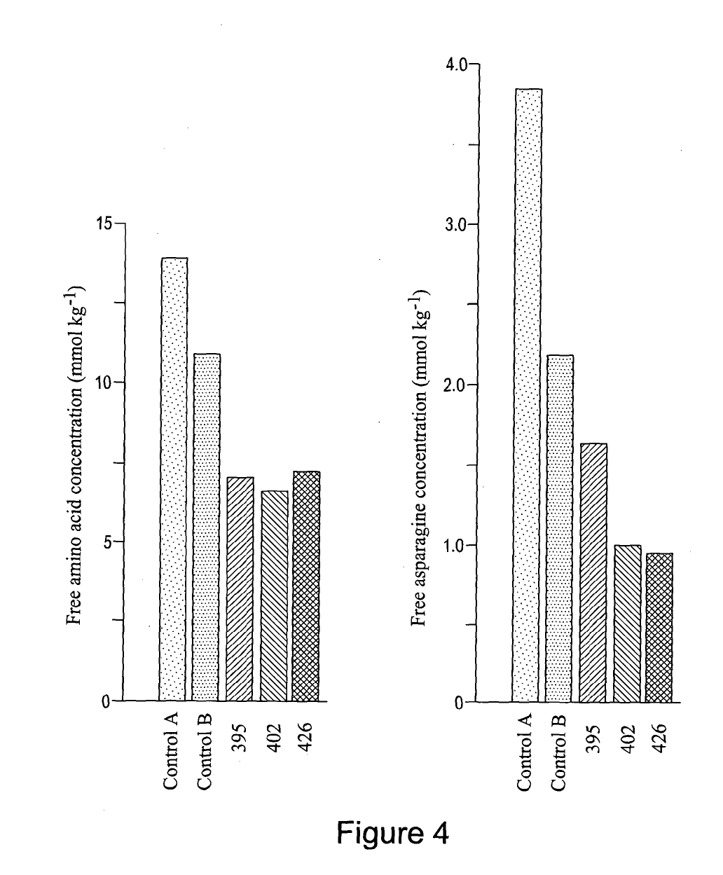

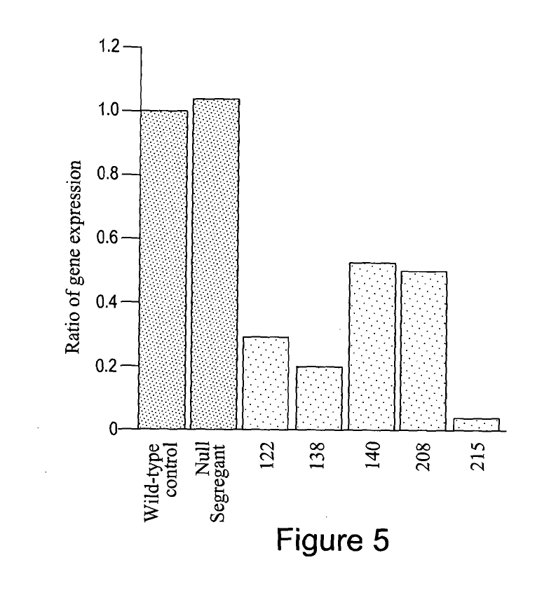

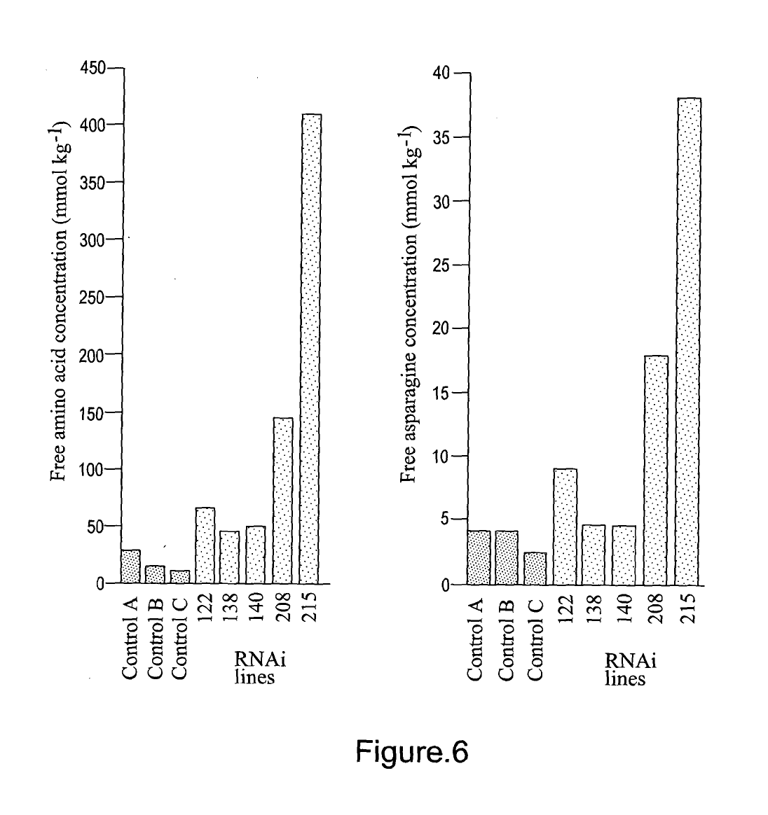

Free amino acid concentrations in the seeds of homozygous T3 plants were measured by Gas Chromatography-Mass Spectrometry (GC-MS) and the results are given in Table 1. Total free amino acid and free asparagine concentrations were significantly reduced (p<0.05) in all three transgenic lines (FIG. 4). In line 426, free asparagine concentration was 0.955 mmol kg⁻¹, compared with an average of 3.00 mmol kg⁻¹ in the controls, a reduction of more than two thirds.

Effects of Manipulating TaGCN2 Gene Expression on Genes of Amino Acid Biosynthesis Under Adequate Nutrient Supply and in Response to Sulphur Deprivation

The transgenic wheat lines over-expressing TaGCN2 were used to investigate the role of TaGCN2 in regulating expression of key genes in amino acid metabolism under conditions of sulphur sufficiency and deficiency. Sulphur deprivation was used to perturb the system in this experiment because it has been shown to cause a massive increase in free amino acid accumulation in wheat, with free asparagine, which can increase 30-fold in concentration in wheat grain, and free glutamine accounting for most of the increase (Muttucumaru et al., 2006; Granvogl et al., 2007; Curtis et al., 2009). The plants were grown in vermiculite, which does not retain nutrients, and feeding was started three weeks after potting. There were two feeding regimes: one set of plants (S+) were watered with ‘complete’ medium containing 1.1 mM MgSO₄ (Muttucumaru et al., 2006; Curtis et al., 2009), a second set (S−) was watered with the same medium containing one tenth the concentration of MgSO₄.

The expression levels of TaGCN2 and a suite of other genes (Table 3) in flag leaves of lines 402 and 426 were compared with those in wheat cv. cadenza controls by real time, quantitative PCR using genes encoding glyceraldehyde 3-phosphate dehydrogenase (GAPDH) and succinate dehydrogenase (SDH) as reference genes. The target gene sequences were identified initially through searches of wheat ESTs using annotated Arabidopsis gene sequences, then checked against rice and Brachypodium genome data. In some cases, additional searches of the wheat genome database were carried out until the full-length gene sequence was obtained. With the exception of aspartate amino transferase (AAT) x, y and z, primers were designed to amplify a product from all three homeologues. In the case of AAT, primer pairs were designed for each different homeologue, but they were called x, y and z because it was not possible to assign the homeologues with certainty to the A, B and D genomes. Primer sequences are given in above under materials and methods.

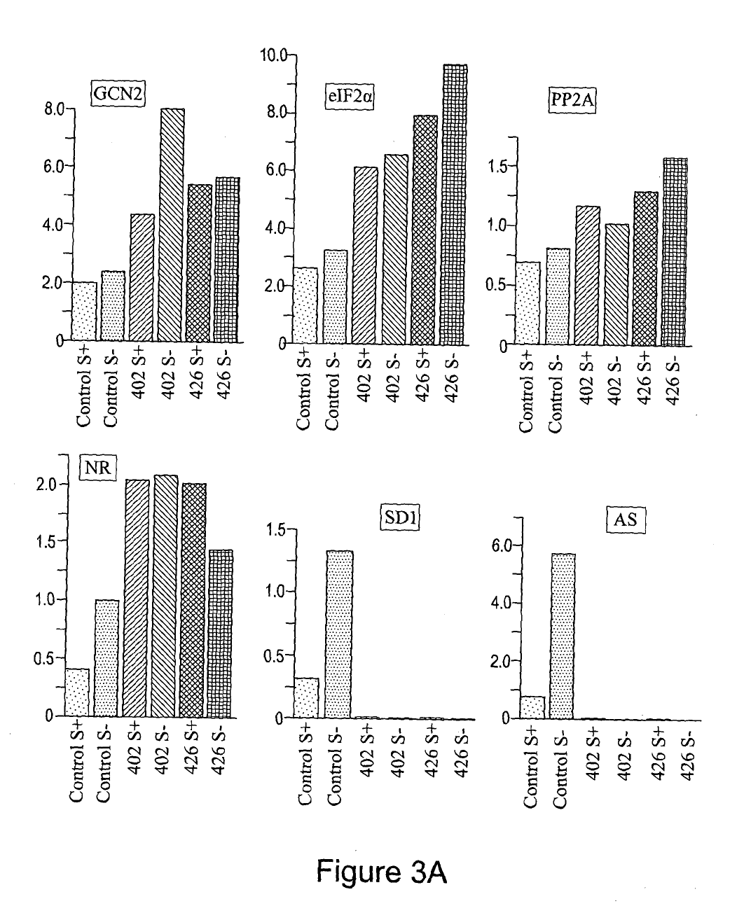

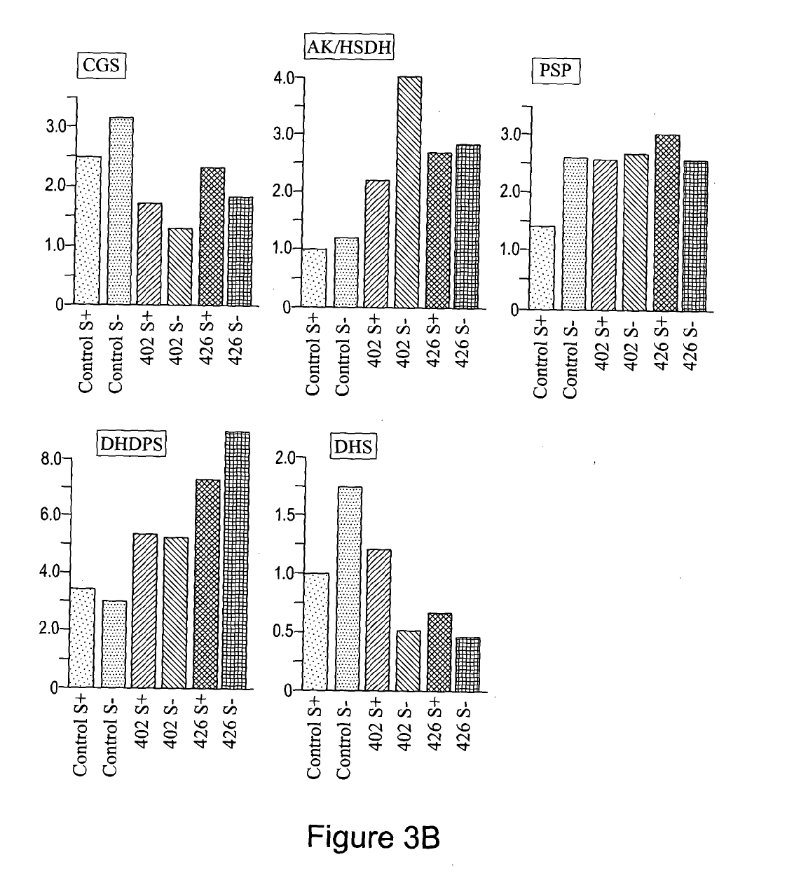

The results of statistical analysis of the gene expression data are given in full in Example 5. The analysis assessed the statistical significance of line and sulphur treatment as main effects and the interaction between these two factors using the F-test. Following an F-test result indicating significant (p<0.05) or marginal (0.05<p<0.10) differences, the least significant difference (LSD) at the 5% level of significance was used to separate pairs of means of interest in the appropriate table of means for each gene. The results for genes which showed significant differences (p<0.05, LSD) between the control plants and both transgenic lines are shown graphically in FIG. 3.

TaGCN2 was confirmed to be significantly (p<0.05) over-expressed in both transgenic lines. Expression of translation initiation factor-2α (eIF2α) and protein phosphatase-2A (PP2A) was also significantly increased. eIF2α is the substrate for phosphorylation by GCN2-type protein kinases, while PP2A dephosphorylates eIF2α, thereby opposing the action of GCN2. The increase in expression of these genes could therefore be interpreted as evidence of the plants compensating for the over-expression of TaGCN2 by producing more substrate and more of the opposing phosphatase. There was also a significant (p<0.05) increase in expression of the nitrate reductase (NR) gene. This is consistent with the finding of Zhang et al. (2008) that expression of a nitrate reductase gene was reduced in an Arabidopsis mutant lacking GCN2, and is therefore further evidence of a role for GCN2 in regulating nitrogen assimilation in plants.

In the plants that were supplied with sulphur there were significantly (p<0.05) higher levels of expression of genes encoding phosphoserine phosphatase (PSP) and dihydropicolinate synthase (DHDPS) in the transgenic lines compared with controls, while expression of an asparagine synthetase (AS1) gene and genes encoding cystathionine gamma-synthase (CGS) and sulphur-deficiency-induced-1 (SDI1) were all significantly (p<0.05) lower. SDI1 is involved in the utilisation of stored sulfate pools under S-limiting conditions and is used as a marker for sulphur deficiency (Howarth et al., 2009), while CGS is involved in the synthesis of the sulphur-containing amino acids, cysteine and methionine, as well as other aspects of sulphur metabolism.

This apparent link with sulphur was dramatically confirmed by the analysis of the sulphur-deprived plants. In the control lines, the expression of genes encoding SDI1 and AS1 increased significantly (p<0.05) (FIG. 4, Example 5), whereas in the TaGCN2 over-expressing lines there was no increase in expression. The expression of two other genes, encoding aspartate kinase/homoserine dehydrogenase (AK/HSDH) and 3-deoxy-D-arabino-heptulosonate-7-phosphate synthase (DHS) (note the alternative abbreviation of DAHP), was significantly (p<0.05) lower in the transgenic lines than in controls under sulphur deprivation. AK/HSDH is a bifunctional enzyme but the phosphorylation of aspartate by its aspartate kinase (AK) activity is the first step in methionine, lysine and threonine synthesis. DHS is involved in the early stages of aromatic amino acid synthesis. Expression of PSP in the control plants increased significantly (p<0.05) in response to sulphur deprivation to the levels seen in the over-expressing plants, which did not change in response to sulphur. In other words, over-expression of TaGCN2 resulted in expression of PSP being at the levels seen in sulphur-deprived control plants whether sulphur was supplied or not.

For the genes encoding AATx and y, there was a significant difference (p<0.05) in expression between the control and line 426, but no significant difference between the control and line 402. There was no significant difference (p>0.10) in expression of AATz, alanine amino transferase (AlaAT), acetolactate synthase (ALS), histidinol dehydrogenase (HDH), or phosphorbosylanthranilate transferase (PAT). The expression of a gene encoding a 14-3-3 protein that interacts with nitrate reductase showed a marginally significant (p<0.10) response to sulphur but was not affected by GCN2 over-expression.

### Example 4

### Yield

The grain yield and 1000 grain weight of the TaGCN2 over-expressing lines was measured and compared with controls. Yield, 1000 grain weight, carbon and nitrogen content of transgenic wheat lines over-expressing TaGCN2 compared with controls. The means over the control and transgenic lines are given in bold with SEs:

| | Yield | 1000 | Nitrogen | Carbon | |
|---|---|---|---|---|---|
| | per | grain | content | content | |
| | plant | weight | (% dry | (% dry | C:N |
| Line | (g) | (g) | weight) | weight) | ratio |
| Control 1 | 13.5 | 39.8 | 3.5 | 45.9 | 13 |
| (wild-type) | | | | | |
| Control 2 | 13.3 | 42.2 | 3.4 | 45.8 | 14 |
| (wild-type) | | | | | |
| Control 3 | 10.8 | 50.8 | 3.5 | 45.7 | 13 |
| (null | | | | | |
| segregant) | | | | | |
| Control 4 | 21.8 | 46.7 | 2.0 | 45.4 | 22 |
| (null | | | | | |
| segregant) | | | | | |
| Control 5 | 25.4 | 45.6 | 2.0 | 45.4 | 22 |
| (null | | | | | |
| segregant) | | | | | |
| Control 6 | 27.6 | 40.3 | 2.0 | 45.3 | 23 |
| (wild-type) | | | | | |
| Control | 18.7 ± 2.9 | 44.2 ± 1.7 | 2.7 ± 0.3 | 45.6 ± 0.1 | 17.8 ± 2.0 |
| mean | | | | | |
| 395 | 19.6 | 55.7 | 2.6 | 45.4 | 17 |
| 402 | 28.8 | 44.7 | 1.9 | 45.3 | 24 |
| 426 | 28.0 | 41.6 | 2.1 | 45.5 | 22 |
| Transgenic | 25.5 ± 2.9 | 47.3 ± 4.3 | 2.2 ± 0.2 | 45.4 ± 0.1 | 21 ± 2.1 |
| mean | | | | | |

As can be seen, there was a trend for the over-expressing lines to yield more grain weight per plant than the control plants, although 1000 grain weight was similar in control and transgenic lines, indicating that there was no difference in the size of individual grains. The nitrogen content of the grain from the transgenic lines was lower than that of controls, while the carbon content was unchanged, meaning that the transgenic lines had a higher ratio of carbon to nitrogen than the controls.

### Example 5

### Analysis of Gene Expression in TaGCN2-Overexpressing Lines 402 and 426 Compared with Wild-Type Wheat Cv. Cadenza Grown with Sulfur Supplied (S+) or Withheld (S−)

Gene expression was analysed by quantitative real-time polymerase chain reaction. There were three biological replicates (leaf tissue samples) for each line by sulfur treatment combination. Two genes, encoding GAPDH and SDH, were used as reference genes for all the other genes. The stability of these genes across the line by sulfur treatment combinations was checked to confirm that they were suitable for this role. The normalised relative quantities (NRQ) for all the genes were calculated. Analysis of variance (ANOVA) was applied to the log (to base 2) transformed inverse of the NRQ data. This transformation ensured homogeneity of variance across the line by sulfur treatment combinations and effectively provided values back on the ct-scale. Therefore, as for ct values, a low log₂(1/NRQ) indicates a high gene expression whereas a high log₂(1/NRQ) indicates low gene expression. The analysis assessed the statistical significance of line and sulfur treatment main effects and the interaction between these two factors using the F-test. Following an F-test result indicating significant (p<0.05) or marginal (0.05<p<0.10) differences, the least significant difference (LSD) at the 5% level of significance was used to separate pairs of means of interest in the appropriate table of means for each gene. Details of the genes analysed are given in the below Gene Table. Primer sequences are given under materials and methods.

The table below shows the p-values from the ANOVA for the result of the F-test on main effects and interactions between the line and sulfur treatment factors for the genes. The genes and p-values in bold indicate that significant (p<0.05) or marginal (0.05<p<0.1) differences between means should be investigated. Also shown are the value of residual variance (s²) and the degrees of freedom (df).

Gene Table

| | | | Line. | s², |
|---|---|---|---|---|
| Gene | Line | Sulfur | Sulfur | residual df |
| GAPDH | 0.166 | 0.960 | 0.727 | 0.1261, 12 |
| GAPDH2 | 0.131 | 0.772 | 0.952 | 0.1887, 12 |
| SDH | 0.166 | 0.960 | 0.727 | 0.1261, 12 |
| AATx-02 | 0.006 | 0.926 | 0.948 | 0.1294, 12 |
| AATy-01 | 0.080 | 0.862 | 0.489 | 0.2198, 12 |
| AATz-01 | 0.237 | 0.967 | 0.518 | 0.08958, 12 |
| AK/HSDH-01 | 0.131 | 0.292 | 0.084 | 0.1932, 12 |
| AK/HSDH-02 | 0.737 | 0.912 | 0.223 | 0.1838, 12 |
| AlaAT-02 | 0.397 | 0.167 | 0.215 | 0.2848, 12 |
| ALS-02 | 0.030 | 0.381 | 0.790 | 0.7779, 12 |
| AS1-03 | <0.001 | 0.464 | 0.071 | 6.131, 12 |
| AS1-04 | <0.001 | 0.455 | 0.169 | 8.030, 11 |
| CGS-01 | 0.026 | 0.645 | 0.207 | 0.1838, 12 |
| DHDPS-01 | 0.058 | 0.256 | 0.717 | 0.8549, 12 |
| DHDPS-03 | 0.012 | 0.607 | 0.837 | 0.3534, 12 |
| DHS2-02 | 0.006 | 0.566 | 0.040 | 0.2601, 12 |
| eIF2a-01a | <0.001 | 0.800 | 0.561 | 0.3162, 12 |
| eIF2a-01b | <0.001 | 0.503 | 0.640 | 0.09902, 12 |
| GCN2-02 | 0.001 | 0.296 | 0.583 | 0.3432, 12 |
| GCN2-03 | 0.001 | 0.589 | 0.872 | 0.5994, 12 |
| HDH-01 | 0.278 | 0.344 | 0.601 | 2.400, 12 |
| NR-01 | <0.001 | 0.517 | 0.155 | 0.3102, 12 |
| PAT1 | 0.616 | 0.726 | 0.951 | 0.1285, 12 |
| PP2A-02a | 0.001 | 0.315 | 0.432 | 0.1877, 12 |
| PP2A-02b | 0.001 | 0.532 | 0.502 | 0.1349, 12 |
| PSP | 0.076 | 0.138 | 0.071 | 0.1845, 12 |
| sdi1-01 | <0.001 | 0.434 | 0.055 | 2.256, 12 |
| sdi1-02 | 0.002 | 0.101 | 0.414 | 2.885, 9 |
| 14-3-3 NR 02 | 0.999 | 0.062 | 0.914 | 0.9885, 11 |

From these results the relevant means tables, on the log₂(1/NRQ) scale, can be considered for comparison of overall line, or overall sulfur, or line by sulfur interaction as appropriate using the LSD (5%) values. These tables are given below.

| AATx-02 Line | Control | L402 | L426 |
|---|---|---|---|
| | 1.270 | 0.888 | 0.441 |

SED = 0.2077, 12 df, LSD(5%) = 0.4526
Control significantly different (p < 0.05) from L426.

| | | | |
|---|---|---|---|
| AATy-01 Line | Control | L402 | L426 |
| | 1.54 | 1.18 | 0.86 |

SED = 0.271, 12 df, LSD(5%) = 0.590
Control significantly different (p < 0.05) from L426.

| | | | |
|---|---|---|---|
| AK/HSDH-01 Line | | S+ | S− |
| | Control | 2.35 | 1.44 |
| | L402 | 2.23 | 2.55 |
| | L426 | 1.96 | 1.88 |

SED = 0.359, 12 df, LSD(5%) = 0.782
Control S+ significantly different (p < 0.05) from control S−.
Control S− significantly different (p < 0.05) from L402 S−.

| | | | |
|---|---|---|---|
| ALS-02 Line | Control | L402 | L426 |
| | −0.02 | −0.94 | −1.58 |

SED = 0.509, 12 df, LSD(5%) = 1.110
Control significantly different (p < 0.05) from L426.

| | | | |
|---|---|---|---|
| AS1-03 Line | | S+ | S− |
| | Control | 2.49 | −2.55 |
| | L402 | 9.53 | 9.89 |
| | L426 | 7.97 | 10.00 |

SED = 2.022, 12 df, LSD(5%) = 4.405
Control significantly different (p < 0.05) from L402 and L426 in S+ and
S−. AS1-03 expression increases for control, when moving from S+ to S−
(p < 0.05), but no significant difference (p > 0.05) for L402 and L426
when comparing S+ to S−.

| | | | |
|---|---|---|---|
| AS1-04 Line | Control | L402 | L426 |
| | 0.34 | 9.96 | 11.57 |

SED = 1.636, 11 df, LSD(5%) = 3.601
Control significantly different (p < 0.05) from L402 and L426.

| | | | |
|---|---|---|---|
| CGS-01 Line | Control | L402 | L426 |
| | −1.41 | −0.62 | −1.04 |

SED = 0.248, 12 df, LSD(5%) = 0.539
Control significantly different (p < 0.05) from L402 and L426.

| | | | |
|---|---|---|---|
| DHDPS-01 Line | Control | L402 | L426 |
| | 1.43 | 0.25 | 0.12 |

SED = 0.534, 12 df, LSD(5%) = 1.163
Control significantly different (p < 0.05) from L402 and L426.

| | | | |
|---|---|---|---|
| DHDPS-03 Line | Control | L402 | L426 |
| | 1.63 | 1.00 | 0.39 |

SED = 0.343, 12 df, LSD(5%) = 0.748
Control significantly different (p < 0.05) from L426.

| | | | |
|---|---|---|---|
| DHS2-02 Line | | S + | S− |
| | Control | 0.08 | −0.77 |
| | L402 | −0.17 | 0.51 |
| | L426 | 0.52 | 1.13 |

SED = 0.416, 12 df, LSD(5%) = 0.907
Control significantly different (p < 0.05) from L402 and L426 in S− only.

| | | | |
|---|---|---|---|
| eIF2a-01a Line | Control | L402 | L426 |
| | 5.19 | 4.11 | 3.43 |

SED = 0.325, 12 df, LSD(5%) = 0.707
Control significantly different (p < 0.05) from L402 and L426.

| | | | |
|---|---|---|---|
| eIF2a-01b Line | Control | L402 | L426 |
| | 5.189 | 3.961 | 3.515 |

SED = 0.1817, 12 df, LSD(5%) = 0.3958
Control significantly different (p < 0.05) from L402 and L426.

| | | | |
|---|---|---|---|
| GCN2-02 Line | Control | L402 | L426 |
| | 5.64 | 4.23 | 4.20 |

SED = 0.338, 12 df, LSD(5%) = 0.737
Control significantly different (p < 0.05) from L402 and L426.

| | | | |
|---|---|---|---|
| GCN2-03 Line | Control | L402 | L426 |
| | 6.99 | 5.75 | 4.77 |

SED = 0.447, 12 df, LSD(5%) = 0.974
Control significantly different (p < 0.05) from L402 and L426.

| | | | |
|---|---|---|---|
| NR-01 Line | Control | L402 | L426 |
| | 7.21 | 5.60 | 5.97 |

SED = 0.322, 12 df, LSD(5%) = 0.701
Control significantly different (p < 0.05) from L402 and L426.

| | | | |
|---|---|---|---|
| PP2A-02a Line | Control | L402 | L426 |
| | 0.65 | −0.31 | −0.54 |

SED = 0.250, 12 df, LSD(5%) = 0.545
Control significantly different (p < 0.05) from L402 and L426.

| | | | |
|---|---|---|---|
| PP2A-02b Line | Control | L402 | L426 |
| | 0.557 | −0.103 | −0.475 |

SED = 0.2120, 12 df, LSD(5%) = 0.4620
Control significantly different (p < 0.05) from L402 and L426.

| | | | |
|---|---|---|---|
| PSP-01 Line | | S + | S− |
| | Control | 2.93 | 1.96 |
| | L402 | 2.04 | 1.74 |
| | L426 | 1.75 | 2.06 |

SED = 0.351, 12 df, LSD(5%) = 0.764
Control S+ significantly different (p < 0.05) from control S−.
Control S+ significantly different (p < 0.05) from L402 and L426 in
S+ only.
L402 and L426 not significantly different (p > 0.05) in S+ and S−.

| | | | |
|---|---|---|---|
| SDI1-01 Line | | S + | S− |
| | Control | 9.71 | 6.41 |
| | L402 | 12.50 | 13.50 |
| | L426 | 12.92 | 13.50 |

SED = 1.226, 12 df, LSD(5%) = 2.672
Control significantly different (p < 0.05) from L402 and L426 in both S+
and S−.
Control S+ significantly different (p < 0.05) from control S−.
L402 and L426 not significantly different (p > 0.05) in S+ and S−.

| | | | |
|---|---|---|---|
| SDI1-02 Line | Control | L402 | L426 |
| | 6.34 | 10.88 | 10.56 |

SED = 0.981, 9 df, LSD(5%) = 2.218
Control significantly different (p < 0.05) from L402 and L426.

| | | |
|---|---|---|
| 14-3-3 NR | S+ | S− |
| | 8.04 | 7.06 |

SED = 0.469, 11 df, p = 0.062

### Example 6

### Potato Plants Overexpressing a GCN2

Arabidopsis GCN2 (AtGCN2) has been ligated in the sense orientation downstream of a potato patatin promoter and the construct is being introduced into potato (cv Pentland Dell) by Agrobacterium-mediated genetic modification. Transgenic potato lines over-expressing AtGCN2 are selected and analysed for tuber yield and composition, including free amino acid and sugar concentration. Acrylamide formation on heating is determined.

### Example 7

### Plant GCN2 Accession Numbers

For the practice of this invention in a wide variety of crops, the following GCN2 analogs are available for use according to this invention.

| | |
|---|---|
| XM_002446674.1 | Sorghum bicolor hypothetical protein, mRNA |
| NM_001059713.1 | Oryza sativa Japonica Group Os04g0492600 (Os04g0492600) |
| | mRNA, partial cds |
| AK072226.1 | Oryza sativa Japonica Group cDNA clone: J013168I08, full insert |
| | sequence |
| NM_001059712.1 | Oryza sativa Japonica Group Os04g0492500 (Os04g0492500) |
| | mRNA, partial cds |
| AK067292.1 | Oryza sativa Japonica Group cDNA clone: J013105J09, full insert |
| | sequence |
| CR855211.1 | Oryza sativa genomic DNA, chromosome 4, BAC clone: H0425E08, |
| | complete sequence |
| AL512542.2 | Oryza sativa genomic DNA, chromosome 4, BAC clone: H0522A01, |
| | complete sequence |
| AL662938.3 | Oryza sativa genomic DNA, chromosome 4, BAC clone: |
| | OJ990528_30, complete sequence |
| AL606629.3 | Oryza sativa genomic DNA, chromosome 4, BAC clone: |
| | OSJNBb0091E11, complete sequence |
| XM473001 | Oryza sativa |
| NM_115803.2 | Arabidopsis thaliana non-specific serine/threonine protein kinase |
| | (GCN2) mRNA, complete cds |
| AJ459823.1 | Arabidopsis thaliana mRNA for GCN2 homologue (gcn2 gene) |
| NM_001203206.1 | Arabidopsis thaliana non-specific serine/threonine protein kinase |
| | (GCN2) mRNA, complete cds |
| XM_002876456.1 | Arabidopsis lyrata subsp. lyrata kinase family protein, mRNA |
| XM_002264803.1 | PREDICTED: Vitis vinifera hypothetical protein LOC100249259 |
| | (LOC100249259), mRNA |
| CP002686.1 | Arabidopsis thaliana chromosome 3, complete sequence |
| AL356014.1 | Arabidopsis thaliana DNA chromosome 3, BAC clone F25L23 |
| XM_002533398.1 | Ricinus communis eif2alpha kinase, putative, mRNA |
| AM424984.2 | Vitis vinifera contig VV78X255687.38, whole genome shotgun |
| | sequence |
| AM485340.2 | Vitis vinifera contig VV78X274751.7, whole genome shotgun |
| | sequence |
| AM460663.2 | Vitis vinifera contig VV78X061118.8, whole genome shotgun |
| | sequence |

### REFERENCES

Chang, L. Y., Yang, W. Y., Browning, K. and Roth, D. (1999) Specific in-vitro phosphorylation of plant eIF2α by eukaryotic eIF2α kinases. Plant Molec. Biol. 41, 363-370.
Chang, L. Y., Yang, W. Y. and Roth, D. (2000) Functional complementation by wheat eIF2α in the yeast GCN2-mediated pathway. Biochem. Biophys. Res. Comm. 279, 468-474.
Chang, S. J., Puryea, J. and Cairney, J. (1993) A simple and efficient method for isolating RNA from pine tree. Plant Molec. Biol. Rep. 11, 113-116.
Chen, J. J. and London, I. M. (1995) Regulation of protein synthesis by heme-regulated eIF2α kinase. Trends Biochem. Sci. 20, 105-108.
Christensen, A. H. and Quail, P. H. (1996) Ubiquitin promoter-based vectors for high-level expression of selectable and/or screenable marker genes in monocotyledonous plants. Transgenic Res. 5, 213-218.
Curtis, T. Y., Muttucumaru, N., Shewry, P. R., Parry, M. A., Powers, S. J., Elmore, J. S., Mottram, D. S., Hook, S. and Halford, N. G. (2009) Evidence for genetic and environmental effects on free amino acid levels in wheat grain: implications for acrylamide formation during processing. J. Agric. Food Chem. 57, 1013-1021.
Curtis, T. Y., Powers, S. J., Balagiannis, D., Elmore, J. S., Mottram, D. S., Parry, M. A. J., Raksegi, M., Bedõ, Z., Shewry, P. R. and Halford, N. G. (2010) Free amino acids and sugars in rye grain: implications for acrylamide formation. J. Agric. Food Chem. 58, 1959-1969.
Friedman, M. (2003) Chemistry, biochemistry and safety of acrylamide. A review. J. Agric. Food Chem. 51, 4504-4526.
Granvogl, M., Wieser, H., Koehler, P., von Tucher, S. and Schieberle P (2007) Influence of sulphur fertilization on the amounts of free amino acids in wheat. Correlation with baking properties as well as with 3-aminopropionamide and acrylamide generation during baking. J. Agric. Food Chem. 55, 4271-4277.
Guyer, D., Patton, D. and Ward, E. (1995) Evidence for cross-pathway regulation of metabolic gene expression in plants. Proc. Natl. Acad. Sci. USA 92, 4997-5000.
Halford, N. G. (2006) Regulation of carbon and amino acid metabolism: roles of sucrose nonfermenting-1-related protein kinase-1 and general control nonderepressible-2-related protein kinase. Adv. Bot. Res. Inc. Adv. Plant Path. 43, 93-142.
Halford, N. G., Curtis, T. Y., Muttucumaru, N., Postles, J. and Mottram, D. S. (2011) Sugars in crop plants. Ann. Appl. Biol. 158, 1-25.
Harding, H. P., Novoa, I., Zhang, Y., Zeng, H., Wek, R., Schapira, M. and Ron, D. (2000) Regulated translation initiation controls stress-induced gene expression in mammalian cells. Molec. Cell 6, 1099-1108.
Hershey, J. W. B. and Merrick W C (2000) The pathway and mechanism of initiation of protein synthesis. In Translational control of gene expression, eds Sonenberg, N., Hershey, J. W. B., Mathews, M. B. (Cold Spring Harbor Laboratory Press, New York), pp. 33-88.
Hinnebusch, A. G. (1992) General and pathway-specific regulatory mechanisms controlling the synthesis of amino acid biosynthetic enzymes in Saccharomyces cerevisiae. In Molecular and Cellular Biology of the Yeast Saccharomyces, Volume 2, Gene Expression, eds Jones, E. W., Pringle, J. R., Broach, J. B. (Cold Spring Harbor Laboratory Press New York), pp. 319-414.
Hinnebusch, A. G. (1994) Translational control of GCN4—an in vivo barometer of initiation factor activity. Trends Biochem. Sci. 19, 409-414.
Hinnebusch, A. G. (1997) Translational regulation of yeast GCN4—A window on factors that control initiator-tRNA binding to the ribosome. J. Biol. Chem. 272, 21661-21664.
Hinnebusch, A. G. (2005) Translational regulation of GCN4 and the general amino acid control of yeast. Ann. Rev. Microbiol. 59, 407-450.
Howarth, J. R., Parmar, S., Barraclough, P. B. and Hawkesford, M. J. (2009) A sulphur-deficiency induced gene, sdi1, involved in the utilisation of stored sulphate pools under S-limiting conditions has potential as a diagnostic indicator of S-nutritional status. Plant Biotech. J. 7, 200-209.
Jefferson, R. A. (1987) Assaying chimeric genes in plants: The GUS gene fusion system. Plant Molec. Biol. Rep. 5, 387-405.
Kaufman, R. J. (1999) Stress signaling from the lumen of the endoplasmic reticulum: Coordination of gene transcriptional and translational controls. Genes Dev. 13, 1211-1233.
Lamacchia, C., Shewry, P. R., Di Fonzo, N., Forsyth, J. L., Harris, N., Lazzeri, P. A., Napier, J. A., Halford, N. G. and Barcelo, P. (2001). Endosperm-specific activity of a storage protein gene promoter in transgenic wheat seed. Journal of Experimental Botany 52, 243-250.
Lageix, S., Lanet, E., Pouch-Pelissier, M. N., Espagnol, M. C., Robaglia, C., Deragon, J. M. and Pelissier, T. (2008) Arabidopsis eIF2α kinase GCN2 is essential for growth in stress conditions and is activated by wounding. BMC Plant Biol. 8, 134-142.
Lea, P. J., Sodek, L., Parry, M. A., Shewry, P. R. and Halford, N. G. (2007) Asparagine in plants. Ann. Appl. Biol. 150, 1-26.
Mascarenhas, C., Edwards-Ingram, L. C., Zeef, L., Shenton, D., Ashe, M. P. and Grant, C. M. (2008) Gcn4 is required for the response to peroxide stress in the yeast Saccharomyces cerevisiae. Molec. Biol. Cell 19, 2995-3007.
McElroy, D., Zhang, W. G., Cao, J. and Wu, R. (1990) Isolation of an efficient actin promoter for use in rice transformation. Plant Cell 2, 163-171.
Mitchell, R. A. C., Castells-Brooke, N., Taubert, J., Verrier, P. J., Leader, D. J. and Rawlings, C. J. (2007) Wheat Estimated Transcript Server (WhETS): a tool to provide best estimate of hexaploid wheat transcript sequence. Nucl. Acids Res. 35, W148-W151.
Mottram, D. S., Wedzicha, B. L. and Dodson, A. T. (2002) Acrylamide is formed in the Maillard reaction. Nature 419, 448-449.
Mottram, D. S. (2007) The Maillard reaction: source of flavour in thermally processed foods. In Flavours and Fragrances: Chemistry, Bioprocessing and Sustainability, ed Berger, R. G. (Springer-Verlag, Berlin, Heidelburg), pp. 269-284.
Muttucumaru, N., Halford, N. G., Elmore, J. S., Dodson, A. T., Parry, M., Shewry, P. R. and Mottram, D. S. (2006) The formation of high levels of acrylamide during the processing of flour derived from sulfate-deprived wheat. J. Agric. Food Chem. 54, 8951-8955.
Nanduri, S., Rahman, F., Williams, B. R. G., and Qin, J. (2000) A dynamically tuned double-stranded RNA binding mechanism for the activation of antiviral kinase PKR. EMBO J. 19, 5567-5574.
Natarajan, K., Meyer, M. R., Jackson, B. M., Slade, D., Roberts, C., Hinnebusch, A. G. and Marton, M. J. (2001) Transcriptional profiling shows that Gcn4p is a master regulator of gene expression during amino acid starvation in yeast. Molec. Cellular. Biol. 21, 4347-4368.
Noctor, G., Veljovic-Jovanovic, S. and Foyer, C. H. (2000) Peroxide processing in photosynthesis: antioxidant coupling and redox signalling. Philos. Trans. R. Soc. London Ser. B—Biol. Sci. 355, 1465-1475.
Ramakers, C., Ruijter, J. M., Deprez, R. H. and Moorman, A. F. (2003) Assumption-free analysis of quantitative real-time polymerase chain reaction (PCR) data. Neurosci. Letts 339, 62-66.
Rocha-Sosa, M., Sonnewald, U., Frommer, W., Stratmann, M., Schell, J. and Willmitzer, L. (1989) Both developmental and metabolic signals activate the promoter of class I patatin gene. EMBO J. 8, 23-29.
Sikalidis, A. K. and Stipanuk, M. H. (2010) Growing rats respond to a sulphur amino acid-deficient diet by phosphorylation of the a subunit of eukaryotic initiation factor 2 heterotrimeric complex and induction of adaptive components of the integrated stress response. J. Nutr. 140, 1080-1085.
Sparks, C. A. and Jones, H. D. (2009) Biolistics transformation of wheat. Methods Molec. Biol. 478, 71-92.
Stadler, R. H., Blank, I., Varga, N., Robert, F., Hau, J., Guy, P. A., Robert, M. C. and Riediker, S. (2002) Acrylamide from Maillard reaction products. Nature 419, 449-450.
Tareke, E., Rydberg, P., Karlsson, P., Eriksson, S. and Tornqvist, M. (2002) Analysis of acrylamide, a carcinogen formed in heated foodstuffs. J. Agric. Food Chem. 50, 4998-5006.
Wang, H., Liu, D., Sun, J. and Zhang, A. (2005) Asparagine synthetase gene TaASN1 from wheat is up-regulated by salt stress, osmotic stress and ABA. J. Plant Physiol. 162, 81-89.
Wek, R. C., Jackson, B. M. and Hinnebusch, A. G. (1989) Juxtaposition of domains homologous to protein kinases and histidyl transfer RNA synthetases in GCN2 protein suggests a mechanism for coupling GCN4 expression to amino acid availability. Proc. Natl. Acad. Sci. USA 86, 4579-4583.
Wek, S. A., Zhu, S. H. and Wek, R. C. (1995) The Histidyl-tRNA synthetase related sequence in the eIF2α protein kinase GCN2 interacts with tRNA and is required for activation in response to starvation for different amino acids. Molec. Cellular Biol. 15, 4497-4506.
Wek, R. C., Ma, K., Vattem, K., Narasimhan, J., Staschke, K. and Jiang, H. Y. (2003) Regulation of eIF2 kinases in response to endoplasmic reticulum and nutritional stresses. FASEB J. 17, A184.
Wek, R. C., Jiang, H. Y. and Anthony, T. G. (2006) Coping with stress: eIF2 kinases and translational control. Biochem. Soc. Trans. 34, 7-11.
Yang, R. J., Wek, S. A. and Wek, R. C. (2000) Glucose limitation induces GCN4 translation by activation of GCN2 protein kinase. Molec. Cellular Biol. 20, 2706-2717.
Zhang, Y., Dickinson, J. R., Paul, M. J. and Halford, N. G. (2003) Molecular cloning of an Arabidopsis homologue of GCN2, a protein kinase involved in co-ordinated response to amino acid starvation. Planta 217, 668-675.
Zhang, Y., Wang, Y., Kanyuka, K., Parry, M. A. J., Powers, S. J. and Halford, N. G. (2008) GCN2-dependent phosphorylation of eukaryotic translation initiation factor-2α in Arabidopsis. J. Exp. Bot. 59, 3131-3141.
Zhao, J., Williams, C. C. and Last, R. L. (1998) Induction of Arabidopsis tryptophan pathway enzymes and camalexin by amino acid starvation, oxidative stress, and an abiotic elicitor. Plant Cell 10, 359-370.
Zhu, S. H., Sobolev, A. Y. and Wek, R. C. (1996) Histidyl-tRNA synthetase-related sequences in GCN2 protein kinase regulate in vitro phosphorylation of eIF-2. J. Biol. Chem. 271, 24989-24994.

TABLE 1
Free amino acid concentrations (mmol kg⁻¹) in flour produced from grain
of transgenic wheat lines in which gene TaGCN2 was over-expressed, and controls
for line 395 (Control A) and for lines 402 and 426 (Control B). Values are
means of three replicates except for Line 402 (two replicates), with (natural)
log transformed data values in parenthesis, which are used to compare lines
using post-ANOVA t-tests based on the standard error of the difference (SED)
value on 33 degrees of freedom (df). Readings in bold indicate lines significantly
different (p < 0.05) from the respective control.

| | Control A | Control B | Line 395 | Line 402 | Line 426 | SED |
|---|---|---|---|---|---|---|
| Alanine | 0.851 | 0.682 | 0.488 | 0.402 | 0.369 | 0.141^{a} |
| | (−0.17) | (−0.38) | (−0.72) | (−0.91) | (−1.00) | 0.127^{b} |
| Arginine | ND | ND | ND | ND | ND | — |
| Asparagine | 3.83 | 2.17 | 1.62 | 0.991 | 0.955 | 0.137 |
| | (1.34) | (0.77) | (0.49) | (−0.01) | (−0.05) | 0.122 |
| Aspartic acid | 3.07 | 2.12 | 1.90 | 2.03 | 1.943 | 0.175 |
| | (1.12) | (0.74) | (0.64) | (0.71) | (0.57) | 0.157 |
| Cysteine | 0.061 | 0.051 | 0.066 | 0.087 | 0.077 | 0.666 |
| | (−3.11) | (−3.04) | (−2.73) | (−2.48) | (−2.57) | 0.596 |
| Glutamic acid | 1.64 | 1.13 | 0.855 | 0.898 | 0.829 | 0.178 |
| | (0.49) | (0.11) | (−0.16) | (−0.12) | (−0.19) | 0.159 |
| Glutamine | 0.891 | 0.331 | 0.113 | 0.114 | 0.083 | 0.460 |
| | (−0.13) | (−1.18) | (−2.27) | (−2.21) | (−2.55) | 0.412 |
| Glycine | 0.281 | 0.246 | 0.204 | 0.169 | 0.172 | 0.136 |
| | (−1.28) | (−1.40) | (−1.59) | (−1.78) | (−1.76) | 0.122 |
| Histidine | 0.203 | 0.249 | 0.134 | 0.128 | 0.097 | 0.651 |
| | (−1.80) | (−1.55) | (−2.09) | (−2.18) | (−2.35) | 0.583 |
| Isoleucine | 0.121 | 0.132 | 0.060 | 0.067 | 0.063 | 0.162 |
| | (−2.12) | (−2.03) | (−2.82) | (−2.71) | (−2.78) | 0.145 |
| Leucine | 0.219 | 0.229 | 0.118 | 0.093 | 0.093 | 0.146 |
| | (−1.52) | (−1.48) | (−2.14) | (−2.39) | (−2.38) | 0.131 |
| Lysine | 0.278 | 0.300 | 0.196 | 0.137 | 0.139 | 0.349 |
| | (−1.32) | (−1.29) | (−1.67) | (−2.05) | (−1.98) | 0.312 |
| Methionine | 0.020 | 0.032 | 0.016 | 0.021 | 0.018 | 0.374 |
| | (−4.00) | (−3.47) | (−4.19) | (−3.86) | (−4.02) | 0.334 |
| Phenylalanine | 0.114 | 0.105 | 0.073 | 0.095 | 0.086 | 0.165 |
| | (−2.17) | (−2.26) | (−2.62) | (−2.37) | (−2.46) | 0.148 |
| Proline | 0.385 | 0.303 | 0.129 | 0.259 | 0.121 | 0.150 |
| | (−0.958) | (−1.20) | (−2.06) | (−1.35) | (−2.12) | 0.134 |
| Serine | 0.872 | 0.999 | 0.681 | 0.652 | 0.479 | 0.817 |
| | (−0.14) | (−0.08) | (−0.47) | (−0.43) | (−0.78) | 0.578 |
| Threonine | 0.875 | 0.921 | 0.347 | 0.189 | 0.667 | 0.727 |
| | (−0.44) | (−0.17) | (−1.48) | (−1.70) | (−1.09) | 0.650 |
| Tryptophan | 0.135 | 1.34 | 0.641 | 0.851 | 1.37 | 0.171 |
| | (−2.02) | (0.29) | (−0.45) | (−0.17) | (0.31) | 0.153 |
| Tyrosine | 0.125 | 0.127 | 0.101 | 0.108 | 0.096 | 0.268 |
| | (−2.10 | (−2.09) | (−2.32) | (−2.27) | (−2.34) | 0.240 |
| Valine | 0.327 | 0.313 | 0.154 | 0.132 | 0.137 | 0.155 |
| | (−1.12) | (−1.17) | (−1.88) | (−2.03) | (−1.99) | 0.139 |
| TOTAL | 13.4 | 10.8 | 7.23 | 6.79 | 7.32 | 0.169 |
| | (2.60) | (2.38) | (1.98) | (1.91) | (1.99) | 0.152 |

^{a}Comparisons with line 426.
^{b}All other comparisons.

TABLE 2
Free amino acid concentrations (mmol kg⁻¹) in flour produced from grain of transgenic
wheat lines in which TaGCN2 gene expression was reduced by RNA interference, and a null
segregant control. Lines 138 and 140 arose from the same transgenic event, as did lines
208 and 215. Values are means of three replicates with (natural) log transformed data
values in parenthesis, which are used to compare lines using post-ANOVA t-tests based
on the standard error of the difference (SED) value on 28 degrees of freedom (df). Readings
in bold indicate lines significantly different (p < 0.05) from the control.

| | Control | Line 122 | Line 138 | Line 140 | Line 208 | Line 215 | SED |
|---|---|---|---|---|---|---|---|
| Alanine | 3.01 | 3.65 | 3.47 | 4.06 | 9.51 | 21.1 | 0.106 |
| | (−0.42) | (−0.32) | (−0.38) | (−0.21) | (0.64) | (1.44) | |
| Arginine | ND | ND | ND | ND | ND | ND | — |
| Asparagine | 3.9 | 9.37 | 4.6 | 4.77 | 17.5 | 36.5 | 0.166 |
| | (−0.13) | (0.62) | (−0.07) | (−0.05) | (1.25) | (1.99) | |
| Aspartic acid | 10.3 | 16.1 | 15.9 | 21.7 | 12.9 | 29.7 | 0.164 |
| | (0.86) | (1.16) | (1.14) | (1.47) | (0.94) | (1.78) | |
| Cysteine | 0.150 | 0.545 | 0.080 | 0.285 | 0.210 | 0.220 | 0.867 |
| | (−3.54) | (−2.44) | (−4.38) | (−2.88) | (−3.23) | (−3.27) | |
| Glutamic acid | 3.11 | 6.38 | 4.05 | 4.29 | 7.79 | 20.3 | 0.151 |
| | (−0.10) | (0.24) | (−0.25) | (−0.16) | (0.44) | (1.40) | |
| Glutamine | 0.235 | 3.53 | 0.710 | 0.150 | 19.4 | 130 | 0.871 |
| | (−3.28) | (−0.51) | (−1.95) | (−3.54) | (1.33) | (3.25) | |
| Glycine | 1.04 | 1.49 | 1.19 | 1.01 | 3.62 | 7.53 | 0.111 |
| | (−1.58) | (−1.21) | (−1.45) | (−1.60) | (−0.33) | (0.41) | |
| Histidine | 0.265 | 1.41 | 0.49 | 0.57 | 0.42 | 1.8 | 0.678 |
| | (−2.94) | (−1.31) | (−2.71) | (−2.19) | (−2.87) | (−1.14) | |
| Isoleucine | 0.330 | 0.860 | 0.580 | 0.400 | 4.24 | 7.64 | 0.167 |
| | (−2.49) | (−1.76) | (−2.16) | (−2.53) | (−0.17) | (0.42) | |
| Leucine | 0.535 | 1.35 | 0.870 | 0.565 | 6.21 | 12.8 | 0.204 |
| | (−2.10) | (−1.33) | (−1.79) | (−2.19) | (0.22) | (0.94) | |
| Lysine | 0.505 | 1.47 | 0.790 | 0.835 | 2.68 | 8.40 | 1.605 |
| | (−2.51) | (−1.51) | (−1.98) | (−1.88) | (−0.69) | (0.47) | |
| Methionine | 0.080 | 0.295 | 0.105 | 0.110 | 0.460 | 1.34 | 0.522 |
| | (−4.38) | (−2.85) | (−3.87) | (−3.99) | (−2.39) | (−1.32) | |
| Phenylalanine | 0.255 | 0.615 | 0.420 | 0.260 | 2.17 | 4.12 | 0.321 |
| | (−2.68) | (−2.10) | (−2.49) | (−2.96) | (−0.84) | (−0.20) | |
| Proline | 0.710 | 3.89 | 1.380 | 0.545 | 21.8 | 74.2 | 0.160 |
| | (−1.78) | (−0.25) | (−1.30) | (−2.22) | (1.47) | (2.69) | |
| Serine | 0.380 | 1.89 | 0.545 | 0.560 | 9.94 | 24.1 | 0.506 |
| | (−2.35) | (−1.02) | (−2.27) | (−2.22) | (0.67) | (1.57) | |
| Threonine | 0.280 | 1.10 | 0.455 | 0.430 | 3.54 | 8.24 | 0.335 |
| | (−2.80) | (−1.54) | (−2.44) | (−2.47) | (−0.36) | (0.50) | |
| Tryptophan | 5.58 | 9.07 | 10.2 | 8.93 | 19.0 | 6.72 | 0.484 |
| | (−0.14) | (0.51) | (0.69) | (0.55) | (1.31) | (0.28) | |
| Tyrosine | 0.190 | 0.880 | 0.570 | 0.795 | 2.57 | 5.61 | 0.473 |
| | (−3.15) | (−1.83) | (−2.22) | (−1.88) | (−0.68) | (0.10) | |
| Valine | 0.690 | 1.27 | 0.920 | 0.740 | 4.92 | 10.8 | 0.131 |
| | (−1.95) | (−1.38) | (−1.70) | (−1.91) | (−0.02) | (0.77) | |
| TOTAL | 34.7 | 65.2 | 47.4 | 51.0 | 149 | 411 | 0.169 |
| | (1.94) | (2.55) | (2.23) | (2.32) | (3.39) | (4.41) | |

TABLE 3
List of genes that were analysed in transgenic wheat lines over-expressing TaGCN2

| Abbreviation | Full name | Comment |
|---|---|---|
| GAPDH | Glyceraldehyde 3-phosphate | REFERENCE GENE |
| | dehydrogenase | |
| SDH | Succinate dehydrogenase | REFERENCE GENE |
| AAT | Aspartate amino transferase | Responsible for conversion of aspartate and α- |
| | | ketoglutarate to oxaloacetate and glutamate |
| AK/HSDH | Aspartate kinase/ | Bifunctional: AK catalyses phosphorylation of |
| | homoserine dehydrogenase | aspartate, first step in synthesis of methionine, |
| | | lysine and threonine. HSDH participates in |
| | | glycine, serine and threonine metabolism and |
| | | lysine synthesis. |
| AlaAT | Alanine amino transferase | Responsible for transfer of an amino group from |
| | | alanine to α-ketoglutarate to give pyruvate and |
| | | glutamate |
| ALS | Acetolactate synthase | Responsible for first step in synthesis of branched |
| | | chain amino acids: valine, leucine and isoleucine |
| AS1 | Asparagine synthetase | Responsible for the ATP-dependent transfer of |
| | | amino group of glutamine to aspartate to generate |
| | | glutamate and asparagine |
| CGS | Cystathionine gamma- | Involved in methionine, cysteine, selenoamino |
| | synthase | acid and sulfur metabolism |
| DHDPS | Dihydrodipicolinate synthase | Responsible for first unique reaction of lysine |
| | | synthesis |
| DHS2 | 3-deoxy-D-arabino- | Involved in early stages of aromatic amino acid |
| | heptulosonate-7-phosphate | biosynthesis |
| | synthase | |
| eIF2α | Translation initiation factor 2α | Substrate of GCN2 |
| GCN2 | General control non- | Over-expressed in transgenic lines |
| | derepressible | |
| HDH | Histidinol dehydrogenase | Involved in histidine metabolism |
| NR | Nitrate reductase | Responsible for key step in incorporation of |
| | | inorganic nitrogen into amino acids |
| PAT1 | Phosphoribosylanthranilate | Involved in tryptophan biosynthesis |
| | transferase | |
| PP2A | Protein phosphatase 2A | Dephosphorylates eIF2α |
| PSP | Phosphoserine phosphatase | Involved in glycine, serine and threonine |
| | | metabolism |
| SDI1 | Sulfur-deficiency-induced-1 | Marker for sulfur deprivation; unknown function |
| 14-3-3NR | 14-3-3 protein | Involved in interaction with nitrate reductase, |
| | | regulating activity. |

1. A method for producing a plant with a reduced amount of one or more free amino acids, the method comprising introducing and over-expressing a nucleic acid construct encoding GCN2 or a functional variant thereof in the plant.

2. The method of claim 1, wherein the nucleic acid construct encodes a plant GCN2.

3. The method of claim 1, wherein the total amount of free amino acids is reduced.

4. The method of claim 1, wherein the total amount of free asparagine is reduced.

5. The method of claim 1, wherein the method reduces the amount of the one or more free amino acids in the plant by about 10% to 80%.

6. The method of claim 5, wherein the method reduces the amount of the one or more free amino acids in the grain, tuber or other harvestable organ of the plant.

7. The method of claim 1, wherein the plant is a monocot or dicot plant.

8. The method of claim 1, wherein the plant is a crop plant.

9. The method of claim 8, wherein the plant is a wheat, rice, potato, soybean, rye, oat, barley or maize plant.

10. The method of claim 8, wherein the plant is a cereal plant.

11.-12. (canceled)

13. The method of claim 1, wherein the nucleic acid construct further comprises a regulatory sequence.

14. A plant made by the method of claim 1.

15. A transgenic plant with a reduced amount of one or more free amino acids, wherein the plant comprises a nucleic acid construct encoding GCN2 or a functional variant thereof.

16. A method for reducing the amount of acrylamide produced upon processing of harvested plant material, the method comprising:
(a) introducing into and over-expressing in a plant a nucleic acid construct encoding GCN2;
(b) harvesting the plant material for processing.

17. The method of claim 16 wherein the plant produces reduced amounts of free asparagine, other free amino acids, or both.

18. The method of claim 16, wherein the reduced amounts of asparagine or other free amino acids is in the grain, tuber or other harvestable organ of a plant.

19. (canceled)

20. A method for producing grain of improved quality by reducing the formation of carcinogenic chemicals during heat processing the grain, wherein the method comprises introducing into and over-expressing in a grain plant a nucleic acid construct encoding GCN2 or a functional variant thereof.

21.-28. (canceled)
